(19) United States
(12) Patent Application Publication
(10) Pub. No.: US 2013/0323733 A1
(43) Pub. Date: Dec. 5, 2013

(54) METHODS OF ASSESSING A RISK OF DEVELOPING NECROTIZING MENINGOENCEPHALITIS

(71) Applicants: Matthew Huentelman, Phoenix, AZ (US); Scott Schatzberg, Phoenix, AZ (US); Renee Barber, Phoenix, AZ (US); Jason Corneveaux, Phoenix, AZ (US)

(72) Inventors: Matthew Huentelman, Phoenix, AZ (US); Scott Schatzberg, Phoenix, AZ (US); Renee Barber, Phoenix, AZ (US); Jason Corneveaux, Phoenix, AZ (US)

(73) Assignee: TRANSLATIONAL GENOMICS RESEARCH INSTITUTE, Phoenix, AZ (US)

(21) Appl. No.: 13/905,109

(22) Filed: May 29, 2013

Related U.S. Application Data
(60) Provisional application No. 61/652,732, filed on May 29, 2012.

(51) Int. Cl. C12Q 1/68

(52) U.S. Cl. CPC C12Q 1/6883
USPC 435/6.11; 536/23.1

(57) ABSTRACT

Methods of using single nucleotide polymorphisms (SNPs), SNP haplotype block, and haplotype to predict whether or not a subject will develop necrotizing meningoencephalitis (NME) and probe sets that facilitate those methods are disclosed. In preferred forms, the subject is a canine species.

### CROSS REFERENCE

This application is related to and claims the priority benefit of U.S. provisional application 61/652,732, filed on May 29, 2012, the teachings and content of which are incorporated by reference herein.

### FIELD OF THE INVENTION

The present invention provides markers, including SNPs, haplotype blocks, and indel variations, that are associated with the risk of developing Necrotizing Meningoencephalitis in canines, particularly, Pugs.

### BACKGROUND OF THE INVENTION

Necrotizing meningoencephalitis (NME) is a non-suppurative inflammatory disorder of the canine central nervous system. Overrepresented in Pug dogs, NME also occurs in other small breeds including the Maltese and Chihuahua. The etiology of NME is unknown but non-Mendelian inheritance has been demonstrated in Pug dogs, suggesting a role for genetic risk factors in the development of disease.

As an idiopathic inflammatory disorder of the central nervous system (CNS), NME primarily affects young to middle aged toy breed dogs. NME shares clinical similarities with atypical variants of multiple sclerosis (MS) in humans. Inflammation in NME is characterized by mixed mononuclear cell infiltrates within the cerebral hemispheres and cortical leptomeninges with common clinical signs including seizures, depression, behavior change, circling and visual deficits. Similar to severe non-prototypical forms of MS, such as the Marburg variant, NME is overrepresented in females, is rapidly progressive, and often carries a grave prognosis despite aggressive immunosuppressive treatment.

NME initially was identified in Pug dogs in the late 1960s and is known to have a strong familial association in this breed. Purebred dog populations provide a unique opportunity for mapping genetic traits and recent technological developments have made it possible to leverage dogs as a model for the study of human genetic disease. Dogs and humans share similar physiology with over half of the known canine diseases having a similar phenotype to analogous human diseases. An evaluation of canine NME, a disorder having clinical similarities to atypical, fulminant variants of MS in human, is needed for identifying at risk and affected dogs, allowing development of targeted therapy and identifying similar genetic factors that are associated with the development of rapidly progressive MS in people.

Whether it is a human population or a canine population, the standard for measuring genetic variation among individuals in a population is the haplotype, which is an ordered combination of specific polymorphisms in the genome in a population. Because haplotypes represent the genetic variation across multiple loci, they provide a more accurate and reliable measurement of genetic variation than individual polymorphisms. In many cases, where an individual polymorphism may be found in a variety of genomic backgrounds, i.e., different haplotypes, no definitive coupling was shown between the polymorphism and the causative site for the phenotype (Clark A G, et al. (1998) Am J Hum Genet 63:595-612; Drysdale, et al. (2000) PNAS 97:10483-10488). Thus, there is an unmet need for information on what NME-associated haplotypes exist in the dog population. Since canine NME is a disorder having clinical similarities to MS in humans, canine NME haplotype information would be useful in improving the efficiency and output of NME and MS diagnosis, prognosis, and several steps in the drug discovery and development process, including target validation, identifying lead compounds, and early phase clinical trials of drugs for MS.

### BRIEF SUMMARY OF THE INVENTION

The present invention generally relates to markers associated with the risk of developing Necrotizing Meningoencephalitis in canines, with the markers being selected from SNPs, haplotype blocks and indel variations.

The present invention provides a combination of markers for identifying NME in canine, which comprises one or more haplotype blocks in a DLA class II region of canine chromosome 12, wherein the DLA class II region comprises sequence having at least 80%, more preferably 90%, still more preferably 95%, 96%, 97%, 98%, 99%, 99.5% or 99.9% sequence identity with SEQ ID NO. 1, wherein SEQ ID NO.1 has a start-end position from 4713000 to 8834700. The one or more haplotype blocks is selected from the group consisting of any one of haplotype blocks 1-19 and any combination thereof in Table 3. Further, the DLA class II region of chromosome 12 comprising the nucleic acid sequence of SEQ ID No. 1 may be defined by one or more tagging SNPs. The one or more tagging SNPs is selected from the group consisting of nucleic acid variation at position 5166878=A or G, 5217389=G or A, 5227499=G or A, 5275229=A or T, 5622709=C or A, 5710832=A or G, 5734305=A or G, 5791672=G or A, 5829667=A or G, 5843592=G or C, 5916360=A or G, 5931001=G or A, 5935549=A or G, 5992526=A or G, 6024841=T or A, 6028685=G or A, 6059850=A or G, 6064245=C or A, 6149213=G or A, 6160615=A or C, 6164202=A or G, 6184107=G or A, 6197313=A or C, 6200280=G or A, 6218850=A or G, 6238545=A or G, 6257019=G or A, 6289014=G or A, 6299459=A or G, 6311277=C or A, 6320910=A or G, 6342204=A or C, 6653816=A or G, 6686088=G or A, 6793393=A or G, 6809061=A or G, 6832252=A or G, and 8822596=C or G, for which 5166878=A, 5217389=G, 5227499=G, 5275229=A, 5622709=C, 5710832=A, 5734305=A, 5791672=G, 5829667=A, 5843592=G, 5916360=A, 5931001=G, 5935549=A, 5992526=A, 6024841=T, 6028685=G, 6059850=A, 6064245=C, 6149213=G, 6160615=A, 6164202=A, 6184107=G, 6197313=A, 6200280=G, 6218850=A, 6238545=A, 6257019=G, 6289014=G, 6299459=A, 6311277=C, 6320910=A, 6342204=A, 6653816=A, 6686088=G, 6793393=A, 6809061=A, 6832252=A and 8822596=C are risk alleles associated with developing Necrotizing Meningoencephalitis; wherein the two or more risk alleles are in linkage disequilibrium with one another.

In one example, the combination of markers comprising one or more haplotype blocks in a DLA class II region of canine chromosome 12 contains one or more haplotype blocks that is selected from the group consisting of haplotype blocks 4-8 and 19, and any combination thereof in Table 3. For example, the haplotype block 4 can be identified by one or more tagging SNP selected from the group consisting of nucleic acid variations at position 5166878=A or G, 5217389=G or A, 5227499=G or A, and 5275229=A or T; wherein 5166878=A, 5217389=G, 5227499=G, and 5275229=A are risk alleles associated with developing Necrotizing Meningoencephalitis. The haplotype block 5 can be identified by a tagging SNP having nucleic acid variations at position 5622709=C or A; wherein 5622709=C is a risk allele. The haplotype block 6 can be identified by one or more tagging SNP selected from the group consisting of nucleic acid variations at position 5710832=A or G, 5734305=A or G, 791672=G or A, 5829667=A or G, 5843592=G or C, 5916360=A or G, 5931001=G or A, 5935549=A or G, 5992526=A or G, 6024841=T or A, 6028685=G or A, 6059850=A or G, and 6064245=C or A; wherein 5710832=A, 5734305=A, 5791672=G, 5829667=A, 5843592=G, 5916360=A, 5931001=G, 5935549=A, 5992526=A, 6024841=T, 6028685=G, 6059850=A, and 6064245=C are risk alleles. The haplotype block 7 is identified by one or more tagging SNP selected from the group consisting of nucleic acid variations at position 6149213=G or A, 6160615=A or C, 6164202=A or G, 6184107=G or A, 6197313=A or C, 6200280=G or A, 6218850=A or G, 6238545=A or G, 6257019=G or A, 6289014=G or A, 6299459=A or G, 6311277=C or A, 6320910=A or G, 6342204=A or C; wherein 6149213=G, 6160615=A, 6164202=A, 6184107=G, 6197313=A, 6200280=G, 6218850=A, 6238545=A, 6257019=G, 6289014=G, 6299459=A, 6311277=C, 6320910=A, and 6342204=A are risk alleles associated with developing Necrotizing Meningoencephalitis. The haplotype block 8 is identified by one or more tagging SNP selected from the group consisting of nucleic acid variations at position 6653816=A or G, 6686088=G or A, 6793393=A or G, 6809061=A or G, 6832252=A or G, wherein 6653816=A or G, 6686088=G or A, 6793393=A or G, 6809061=A or G, 6832252=A or G are risk alleles. The haplotype block 19 is identified by a tagging SNP having nucleic acid variations at position 8822596=C or G; wherein 8822596=C is a risk allele associated with developing Necrotizing Meningoencephalitis.

The combination of markers for identifying NME in canine, which comprises one or more haplotype blocks in a DLA class II region of canine chromosome 12, having a start-end position from about 4713000 to about 8834700, may further comprise a haplotype block in STYX region of chromosome 8, wherein the STYX region comprises sequence having at least 80%, more preferably 90%, still more preferably 95%, 96%, 97%, 98%, 99%, 99.5% or 99.9% sequence identity with SEQ ID No. 2, wherein SEQ ID No. 2 has a start-end position from 31736000 to 32225100. This STYX region of chromosome 8 comprises a tagging SNP having nucleic acid variation at position 31971609=A or G, wherein 31971609=A is a risk allele associated with developing Necrotizing Meningoencephalitis.

The combination of markers for identifying NME in canine, which comprises one or more haplotype blocks in a DLA class II region of canine chromosome 12, having a start-end position from about 4713000 to about 8834700, may further comprise a HLA-DPB1 single base deletion variant at position 5608903, wherein the deletion variant comprising SEQ ID NO.26 is associated with developing Necrotizing Meningoencephalitis. The haplotype block 7 can be identified by the HLA-DPB1 single base deletion variant comprising SEQ ID NO.26. The haplotype block 7 may be further identified by the HLA-DPB1 single base deletion variant comprising SEQ ID NO.26 and a tagging SNP comprising nucleic acid variations at position 5622709=C or A; wherein 5622709=C is a risk allele associated with developing Necrotizing Meningoencephalitis. The one or more haplotype blocks of the combination of markers in a DLA class II region of canine chromosome 12 may be detected by genotyping using PCR, sequencing, hybridization, restriction digestion, or any combination thereof. The combinations of the markers as disclosed herein are suitable for canine species selected from a group consisting of Pug, Chihuahua, West Highland White Terrier, Pekingese, Labrador Retriever, Golden Retriever, Beagle, German Shepherd, Dachshund, Yorkshire Terrier, Boxer, Poodle, Shih Tzu, Miniature Schnauzer, Pomeranian, Cocker Spaniel, Rottweiler, Bulldog, Shetland Sheepdog, Boston Terrier, Miniature Pinscher, Maltese, German Shorthaired Pointer, Doberman Pinscher, Siberian Husky, Pembroke Welsh Corgi, Basset Hound, Bichon Frise, and other currently recognized and or yet to be recognized breeds.

The present invention further provides a combination of markers for identifying NME in canine which comprises a HLA-DPB1 single base deletion variant at position 5608903 of canine chromosome 12, and the deletion variant comprising SEQ ID NO.26 is associated with developing Necrotizing Meningoencephalitis. In addition to a HLA-DPB 1 single base deletion variant at position 5608903 of canine chromosome 12, such a combination of markers further comprises one or more tagging SNPs selected from the group consisting of nucleic acid variations at position 5166878=A or G, 5217389=G or A, 5227499=G or A, 5275229=A or T, 5622709=C or A, 5710832=A or G, 5734305=A or G, 5791672=G or A, 5829667=A or G, 5843592=G or C, 5916360=A or G, 5931001=G or A, 5935549=A or G, 5992526=A or G, 6024841=T or A, 6028685=G or A, 6059850=A or G, 6064245=C or A, 6149213=G or A, 6160615=A or C, 6164202=A or G, 6184107=G or A, 6197313=A or C, 6200280=G or A, 6218850=A or G, 6238545=A or G, 6257019=G or A, 6289014=G or A, 6299459=A or G, 6311277=C or A, 6320910=A or G, 6342204=A or C, 6653816=A or G, 6686088=G or A, 6793393=A or G, 6809061=A or G, 6832252=A or G, and 8822596=C or G; for which, 5166878=A, 5217389=G, 5227499=G, 5275229=A, 5622709=C, 5710832=A, 5734305=A, 5791672=G, 5829667=A, 5843592=G, 5916360=A, 5931001=G, 5935549=A, 5992526=A, 6024841=T, 6028685=G, 6059850=A, 6064245=C, 6149213=G, 6160615=A, 6164202=A, 6184107=G, 6197313=A, 6200280=G, 6218850=A, 6238545=A, 6257019=G, 6289014=G, 6299459=A, 6311277=C, 6320910=A, 6342204=A, 6653816=A, 6686088=G, 6793393=A, 6809061=A, 6832252=A; and 8822596=C are risk alleles associated with developing Necrotizing Meningoencephalitis; wherein the two or more risk alleles are in linkage disequilibrium with one another.

The present invention also provides a method of classifying a subject to an NME disease risk group, which comprises the steps of receiving a nucleic acid-containing sample from the subject; detecting the presence of a combination of markers comprising one or more haplotype blocks and tagging SNPs in a DLA class II region of canine chromosome 12 comprising sequences having at least 80%, more preferably 90%, still more preferably 95%, 96%, 97%, 98%, 99%, 99.5% or 99.9% sequence identity with SEQ ID No. 1, wherein SEQ ID No.1 has start-end position from 4713000 to 8834700; and classifying the subject into a risk group based upon the presence of at least one haplotype block, or classifying the subject into a non-risk group based upon the absence of any haplotype block. In the general method, the one or more haplotype blocks is selected from the group consisting of haplotype blocks 1-19 and any combination thereof in Table 3. Further, the one or more tagging SNPs in the general method is selected from the group consisting of nucleic acid variation at position 5166878=A or G, 5217389=G or A, 5227499=G or A, 5275229=A or T, 5622709=C or A, 5710832=A or G, 5734305=A or G, 5791672=G or A, 5829667=A or G, 5843592=G or C, 5916360=A or G, 5931001=G or A, 5935549=A or G, 5992526=A or G, 6024841=T or A, 6028685=G or A, 6059850=A or G, 6064245=C or A, 6149213=G or A, 6160615=A or C, 6164202=A or G, 6184107=G or A, 6197313=A or C, 6200280=G or A, 6218850=A or G, 6238545=A or G, 6257019=G or A, 6289014=G or A, 6299459=A or G, 6311277=C or A, 6320910=A or G, 6342204=A or C, 6653816=A or G, 6686088=G or A, 6793393=A or G, 6809061=A or G, 6832252=A or G, and 8822596=C or G, for which 5166878=A, 5217389=G, 5227499=G, 5275229=A, 5622709=C, 5710832=A, 5734305=A, 5791672=G, 5829667=A, 5843592=G, 5916360=A, 5931001=G, 5935549=A, 5992526=A, 6024841=T, 6028685=G, 6059850=A, 6064245=C, 6149213=G, 6160615=A, 6164202=A, 6184107=G, 6197313=A, 6200280=G, 6218850=A, 6238545=A, 6257019=G, 6289014=G, 6299459=A, 6311277=C, 6320910=A, 6342204=A, 6653816=A, 6686088=G, 6793393=A, 6809061=A, 6832252=A; and 8822596=C are risk alleles associated with developing Necrotizing Meningoencephalitis; wherein the two or more risk alleles are in linkage disequilibrium with one another. The general method provided herein is suitable for canine species selected from a group consisting of Pug, Chihuahua, West Highland White Terrier, Pekingese, Labrador Retriever, Golden Retriever, Beagle, German Shepherd, Dachshund, Yorkshire Terrier, Boxer, Poodle, Shih Tzu, Miniature Schnauzer, Pomeranian, Cocker Spaniel, Rottweiler, Bulldog, Shetland Sheepdog, Boston Terrier, Miniature Pinscher, Maltese, German Shorthaired Pointer, Doberman Pinscher, Siberian Husky, Pembroke Welsh Corgi, Basset Hound, Bichon Frise, and other currently recognized and or yet to be recognized breeds.

One aspect of the present invention provides an isolated nucleic acid molecule selected from the group consisting of: (a) an isolated nucleic acid molecule comprising the sequence of SEQ ID NO:1; (b) an isolated nucleic acid molecule comprising the sequence of SEQ ID NO:2; (c) an isolated nucleic acid molecule comprising a segment of SEQ ID NO:1; (d) an isolated nucleic acid molecule comprising a segment of SEQ ID NO:2; and, (e) an isolated nucleic molecule which is complementary to the isolated nucleic acid molecule of (a), (b), (c), and (d); wherein the isolated nucleic acid molecule comprises one or more markers selected from the group consisting of SNP alleles, haplotype blocks, or gene indel variants that are associated with a risk or non-risk of developing Necrotizing Meningoencephalitis in a subject. The marker is selected from the group consisting of SNPs as listed in FIG. 1 and FIG. 2, haplotype blocks listed in Table 3 and Table 4, and gene indel variants in the DLA II region of canine Chromosome 12 and the STYX region of Chromosome 8.

In some examples, said isolated nucleic acid molecule comprises two or more single nucleotide polymorphisms (SNPs) as listed in FIG. 1 and FIG. 2, wherein the two or more SNPs are in linkage disequilibrium with one another. In one example, the gene indel variant is an HLA-DPB 1 deletion variant represented by SEQ ID NO: 26, and this deletion allele of HLA-DPB 1 is associated with a risk of developing Necrotizing Meningoencephalitis. In another example, the one or more SNPs having loci in the sequence of SEQ ID NO: 1 and SEQ ID NO: 2 are selected from a group consisting of nucleotide variations: 5166878=A or G; 5217389=G or A; 5227499=G or A; 5275229=A or T; 5622709=C or A; 5710832=A or G; 5734305=A or G; 5791672=G or A; 5829667=A or G; 5843592=G or C; 5916360=A or G; 5931001=G or A; 5935549=A or G; 5992526=A or G; 6024841=T or A; 6028685=G or A; 6059850=A or G; 6064245=C or A; 6149213=G or A; 6160615=A or C; 6164202=A or G; 6184107=G or A; 6197313=A or C; 6200280=G or A; 6218850=A or G; 6238545=A or G; 6257019=G or A; 6289014=G or A; 6299459=A or G; 6311277=C or A; 6320910=A or G; 6342204=A or C; 6653816=A or G; 6686088=G or A; 6793393=A or G; 6809061=A or G; 6832252=A or G; 8822596=C or G, on canine Chromosome 12 at the indicated position; and 31971609=A or G on canine Chromosome 8 at the indicated position. Specifically, the one or more SNPs that are associated with a risk of developing Necrotizing Meningoencephalitis are selected from a group consisting of nucleotide: 5166878=A; 5217389=G; 5227499=G; 5275229=A; 5622709=C; 5710832=A; 5734305=A; 5791672=G; 5829667=A; 5843592=G; 5916360=A; 5931001=G; 5935549=A; 5992526=A; 6024841=T; 6028685=G; 6059850=A; 6064245=C; 6149213=G; 6160615=A; 6164202=A; 6184107=G; 6197313=A; 6200280=G; 6218850=A; 6238545=A; 6257019=G; 6289014=G; 6299459=A; 6311277=C; 6320910=A; 6342204=A; 6653816=A; 6686088=G; 6793393=A; 6809061=A; 6832252=A; 8822596=C, on canine Chromosome 12 at the indicated position; and 31971609=A on canine Chromosome 8 at the indicated position. In still another example, the one or more haplotype blocks that are associated with a risk of developing Necrotizing Meningoencephalitis are selected from a group consisting of sequences represented by: SEQ ID NO: 3 at position 4713392-4821633, SEQ ID NO:4 at position 4836721-4923170, SEQ ID NO:5 at position 4938082-5088561, SEQ ID NO:6 at position 5108726-5364188, SEQ ID NO:7 at position 5491709-5672682, SEQ ID NO:8 at position 5710832-6078099, SEQ ID NO:9 at position 6149213-6342204, SEQ ID NO:10 at position 6492201-6982375, SEQ ID NO:11 at position 6992493-7270218, SEQ ID NO:12 at position 7338759-7350261, SEQ ID NO:13 at position 7384390-7643147, SEQ ID NO:14 at position 7725530-7830733, SEQ ID NO:15 at position 7927872-7944953, SEQ ID NO:16 at position 7950821-8158994, SEQ ID NO:17 at position 8208369-8265940, SEQ ID NO:18 at position 8327142-8386063, SEQ ID NO:19 at position 8429601-8533350, SEQ ID NO:20 at position 8546686-8713747, SEQ ID NO:21 at position 8719506-8834652, on canine Chromosome 12; and SEQ ID NO: 22 at position 31,736,206 to 31,795,128, SEQ ID NO: 23 at position 31,866,373 to 31,883,390, SEQ ID NO: 24 at position 31,971,609 to 32,009,283, and SEQ ID NO: 25 at position 32,183,184 to 32,225,068, on canine Chromosome 8. In some examples, the isolated nucleic acid molecule may be in the form of a probe or primer, and the probe or primer hybridizes under stringent conditions to said isolated nucleic acid molecule of claim 1.

Generally, the subject under the detection for the above one or more markers is a canine species selected from a group consisting of Pug, Chihuahua, West Highland White Terrier, Pekingese, Labrador Retriever, Golden Retriever, Beagle, German Shepherd, Dachshund, Yorkshire Terrier, Boxer, Poodle, Shih Tzu, Miniature Schnauzer, Pomeranian, Cocker Spaniel, Rottweiler, Bulldog, Shetland Sheepdog, Boston Terrier, Miniature Pinscher, Maltese, German Shorthaired Pointer, Doberman Pinscher, Siberian Husky, Pembroke Welsh Corgi, Basset Hound, Bichon Frise, and other currently recognized and or yet to be recognized breeds.

Another aspect of the present invention provides a method of classifying a subject to an NME disease risk group. The method comprises (1) receiving a nucleic acid-containing sample from the subject; (2) detecting the presence of one or more markers selected from the group consisting of one or more SNP alleles, haplotype blocks, and gene indel variants that are associated with a risk of developing Necrotizing Meningoencephalitis in the subject, wherein the SNP alleles, haplotype blocks, and gene indel variants are represented by nucleic acid segments in SEQ ID NO:1 and SEQ ID NO:2; and (3) classifying the subject into a risk group based upon the presence of at least one haplotype block, or classifying the subject into a non-risk group based upon the absence of any haplotype block. In one example of performing the general method, the one or more SNP alleles are selected from the group consisting of nucleotide: 5166878=A; 5217389=G; 5227499=G; 5275229=A; 5622709=C; 5710832=A; 5734305=A; 5791672=G; 5829667=A; 5843592=G; 5916360=A; 5931001=G; 5935549=A; 5992526=A; 6024841=T; 6028685=G; 6059850=A; 6064245=C; 6149213=G; 6160615=A; 6164202=A; 6184107=G; 6197313=A; 6200280=G; 6218850=A; 6238545=A; 6257019=G; 6289014=G; 6299459=A; 6311277=C; 6320910=A; 6342204=A; 6653816=A; 6686088=G; 6793393=A; 6809061=A; 6832252=A; 8822596=C, on canine Chromosome 12 at the indicated position; and 31971609=A on canine Chromosome 8 at the indicated position.

In another example of performing the general method, the one or more haplotype blocks are selected from the group consisting of sequences represented by: SEQ ID NO: 3 at position 4713392-4821633, SEQ ID NO:4 at position 4836721-4923170, SEQ ID NO:5 at position 4938082-5088561, SEQ ID NO:6 at position 5108726-5364188, SEQ ID NO:7 at position 5491709-5672682, SEQ ID NO:8 at position 5710832-6078099, SEQ ID NO:9 at position 6149213-6342204, SEQ ID NO:10 at position 6492201-6982375, SEQ ID NO:11 at position 6992493-7270218, SEQ ID NO:12 at position 7338759-7350261, SEQ ID NO:13 at position 7384390-7643147, SEQ ID NO:14 at position 7725530-7830733, SEQ ID NO:15 at position 7927872-7944953, SEQ ID NO:16 at position 7950821-8158994, SEQ ID NO:17 at position 8208369-8265940, SEQ ID NO:18 at position 8327142-8386063, SEQ ID NO:19 at position 8429601-8533350, SEQ ID NO:20 at position 8546686-8713747, SEQ ID NO:21 at position 8719506-8834652, on canine Chromosome 12; and SEQ ID NO: 22 at position 31,736,206 to 31,795,128, SEQ ID NO: 23 at position 31,866,373 to 31,883,390, SEQ ID NO: 24 at position 31,971,609 to 32,009,283, and SEQ ID NO: 25 at position 32,183,184 to 32,225,068, on canine Chromosome 8.

In yet another example of performing the general method, the marker is the gene indel variant HLA-DPB 1 deletion variant represented by SEQ ID NO: 26.

In some marker detection steps in the general method provided, said step may further comprise a method selected from the group consisting of Sanger sequencing, next generation sequencing, pyrosequencing, sequencing by ligation, sequencing by synthesis, single molecule sequencing, pooled and barcoded DNA sequencing, PCR, real-time PCR, quantitative PCR, microarray analysis of genomic DNA, restriction fragment length polymorphism analysis, allele specific ligation, and comparative genomic hybridization. Alternatively, the marker detection step may further comprise microarray analysis of RNA, RNA in situ hybridization, RNAse protection assay, Northern blot, reverse transcription PCR, quantitative PCR, quantitative reverse transcription PCR, quantitative real-time reverse transcription PCR, reverse transcription treatment followed by direct sequencing, flow cytometry, immunohistochemistry, ELISA, Western blot, immunoaffinity chromatography, HPLC, mass spectrometry, protein microarray analysis, PAGE analysis, isoelectric focusing, and 2-D gel electrophoresis.

Generally, the subject upon which the method is performed is a canine species selected from a group consisting of Pug, Chihuahua, West Highland White Terrier, Pekingese, Labrador Retriever, Golden Retriever, Beagle, German Shepherd, Dachshund, Yorkshire Terrier, Boxer, Poodle, Shih Tzu, Miniature Schnauzer, Pomeranian, Cocker Spaniel, Rottweiler, Bulldog, Shetland Sheepdog, Boston Terrier, Miniature Pinscher, Maltese, German Shorthaired Pointer, Doberman Pinscher, Siberian Husky, Pembroke Welsh Corgi, Basset Hound, Bichon Frise, and other currently recognized and or yet to be recognized breeds.

Another aspect of the present invention provides a set of molecular probes used in assessing the risk of developing NME of a subject, which comprises a first probe capable of detecting a first marker selected from the group consisting of a SNP allele listed in FIG. 1 and FIG. 2, a haplotype block in Table 3 and Table 4, and an HLA-DPB1 deletion variant represented by SEQ ID NO: 26; and a second probe, different from the first probe, capable of detecting a second marker selected from the group consisting of a SNP allele listed in FIG. 1 and FIG. 2, a haplotype block in Table 3 and Table 4, and an HLA-DPB1 deletion variant represented by SEQ ID NO: 26, wherein the markers detected by the probes are associated with a high risk of developing NME and wherein the risk group comprises subjects with a high risk of developing NME.

Other aspects and iterations of the invention are described in more detail below.

### BRIEF DESCRIPTION OF THE FIGURES

FIG. 1 depicts the SNPs in the DLA class II region on Chromosome 12, with alternative bases as listed for A_{R}, the allele associated with NME risk, and A_{NR}, the non-risk allele.

FIG. 2 depicts the SNP in the STYX region on Chromosome 8, with alternative base as listed for A_{R}, the allele associated with NME risk, and A_{NR}, the non-risk allele.

FIG. 3 depicts the genome-wide association results for 28 NME cases and 45 controls. FIG. 3 (a) represents Fisher's exact tests were performed to compare SNP allele frequencies and negative log P values were plotted across the genome. The horizontal dotted line represents the threshold for significant association after Bonferroni correction of −log(P)>6.24 with a strong peak on chromosome 12 maintaining genome-wide significance. FIG. 3 (b) represents MaxT 100,000 permutation testing was performed and negative log P values were plotted across the genome. The horizontal dotted line represents the threshold for significant association after permutation testing of −log(P)>1.3 with one SNP on chromosome 8 maintaining permuted significance.

FIG. 4 depicts haplotype analysis of NME-associated DLA II locus on chromosome 12. A 4.1 Mb region located from positions 4,713,392 to 8,834,652 with 19 haplotype blocks generated in Haploview. Negative log P values from single SNP associations were derived from genome-wide analysis after removal of population outliers and individual SNPs were plotted as circles. The horizontal dotted line represents the threshold for significant association after Bonferroni correction of −log(P)>6.24.

FIG. 5 depicts haplotype analysis of NME-associated locus on chromosome 8. A 488 kb region located from positions 31,736,206 to 32,225,068 is shown with 4 haplotype blocks generated in Haploview. Negative log P values from single SNP associations were derived from genome-wide analysis after removal of population outliers and individual SNPs were plotted as circles. The horizontal dotted line represents the threshold for significant association after Bonferroni correction of −log(P)>6.24. The additional shaded region within each haplotype block represents MaxT 100,000 permuted haplotypes.

### DETAILED DESCRIPTION OF THE INVENTION

The disclosure provides a method of assigning a subject to a necrotizing meningoencephalitis (NME) risk group in order to assess the likelihood of the subject being afflicted with the disease. This method can be employed to assess the risk at early stages of disease progression. The method includes providing a biological sample from the subject, detecting a marker in a biological sample, which can be a haplotype associated with NME and assigning the subject to the NME risk group based upon the presence or absence of the haplotype. The method involves directly or indirectly detecting the presence or absence of the marker. Multiple markers disclosed herein may be used in combination to improve the accuracy.

A haplotype refers to a segment of genomic DNA that is characterized by a specific combination of genetic markers (or alleles) arranged along the segment. A marker refers to a sequence characteristic of a particular allele. Detection of the disease also includes detection of the haplotype by SNPs (single nucleotide polymorphisms) or other types of markers for the haplotype, but also indirectly through markers outside the haplotype by leveraging linkage disequilibrium to identify carriers of the haplotype. Examining haplotypes rather than individual SNPs gives much stronger signal in detecting disease association. In addition, haplotypes can represent a combined effect of several sites along the same chromosome that cannot be detected when these sites are tested one by one. In the present invention, in addition to determining a patient's relative risk for NME by haplotype, haplotype blocks, or tag SNPs, the diagnosis may include prescribing therapeutic regimens to treat, prevent or delay onset of NME.

A haplotype may be any combination of one or more closely linked alleles inherited as a unit with little genetic shuffling across generations. An allele includes any form of a particular nucleic acid that may be recognized as a form of the particular nucleic acid on account of its location, sequence, or any other characteristic that may identify it as being a form of the particular gene. Alleles include but need not be limited to forms of a gene (or, broadly, a nucleic acid sequence) that include point mutations, silent mutations, deletions, frame-shift mutations, SNPs, inversions, translocations, heterochromatic insertions, and differentially methylated sequences relative to a reference gene (or, broadly, a wild type nucleic acid sequence), whether alone or in combination. An allele of a gene may or may not produce a functional protein; may produce a protein with altered function, localization, stability, dimerization, or protein-protein interaction; may have overexpression, underexpression or no expression; may have altered temporal or spatial expression specificity. The presence or absence of an allele may be detected through the use of any process known in the art, including using primers and probes designed accordingly for PCR, sequencing, hybridization analyses. An allele may also be called a mutation or a mutant, in view of its wild-type. An allele may be compared to another allele that may be termed a wild type form of an allele. In some cases, the wild type allele is more common than the mutant.

A haplotype may comprise a specific combination of polymorphisms. The polymorphism may be a single nucleotide polymorphism. The genetic sequences of different individuals are remarkably similar. However, when the chromosomes of two humans are compared, on average, there is about one in every 1000 to 1,200 bases of the DNA sequences that differs. As such, one individual might have an A at that location, while another individual has a G, or a person might have extra bases at a given location or a missing segment of DNA. Differences in individual bases are the most common type of genetic variation. These genetic differences are known as single nucleotide polymorphisms (SNPs). SNPs associated with a nearby gene may be used as markers to locate or map the gene. Some SNPs are associated with each other, and may be inherited in blocks.

The difference of a single genetic variance such as a SNP can delineate a distinct haplotype. A “SNP haplotype block” or “haplotype block” is a nucleic acid sequence containing a group of SNPs or polymorphisms that do not appear to recombine independently resulting in reduced genetic variability, but are passed together from generation to generation in variable-length blocks. The combination of polymorphisms, haplotype patterns and haplotype blocks may be referred to as a “haplotype” or “haplotype structure” in a nucleic acid sequence of interest. For example, a haplotype can be a set of SNPs, alleles, or genetic markers on a single chromatid that are genetically linked, and thus, are likely to be inherited as a unit. “Linked”, “linkage”, or “allelic association” means the preferential association of a particular allele or genetic marker with a specific allele or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles. A marker in linkage disequilibrium can be particularly useful in detecting susceptibility to disease (or other phenotype) notwithstanding that the marker itself does not cause the disease. For example, a marker (X) that is not itself a causative element of a disease, but which is in linkage disequilibrium with a gene (including regulatory sequences) (Y) that is a causative element of a phenotype, can be detected to indicate susceptibility to the disease in circumstances in which the gene Y may not have been identified or may not be readily detectable.

Haplotypes are the particular combinations of alleles observed in a population, and are regions over which there is a very small proportion (often less than 5%) of informative SNP pairs showing strong evidence of historical recombination. Haplotype patterns may be disrupted by forces other than recombination, such as, recurrent mutation, gene conversion, or simply genome assembly or genotyping errors, which accounts for the approximate 5% disruption. Generally, directly genotyping pedigrees and using molecular methods, such as allele-specific polymerase chain reaction (AS-PCR) and somatic cell hybrids, in combination with genotyping can unambiguously assign alleles to chromosomes and thus determine the haplotypes. Other methods of determining or constructing a haplotype include statistical inference using various algorithms to process genotype data. Further, two or more alleles likely to be inherited as a unit is termed a haplotype block. Typically, a haplotype block refers to a chromosome region of high linkage disequilibrium and low haplotype diversity, and are regions of low recombination flanked by recombination hotspots (e.g., Cardon, L R and Abecasis, G R, Trends in Genetics, 19(3):135-140 (2003)). When one or more haplotype blocks are associated with a phenotypic trait, the haplotype block serves as a genetic marker represented by a genetic locus comprising one or more linked genetic variations that would be inherited as a unit, more frequently than not, in an individual having the associated phenotypic trait. In each block, a small fraction of single-nucleotide polymorphisms (SNPs), referred to as “tag SNPs,” (also called “tagging SNPs”) can be used to distinguish a large fraction of the haplotypes. The main advantage of using haplotype blocks for phenotype association is that only a few tag SNPs need to be genotyped to represent haplotypes within a block.

Tag SNPs are those markers capturing the haplotype diversity to best represent the genetic variation of a haplotype. As such, genotyping a tag SNP would eliminate the need to genotype other markers in strong linkage disequilibrium with the tag SNP. Tag SNPs may be determined using various algorithms. Different algorithms may result in different sets of tag SNPs because of their various efficiency which is affected by the specificity, size of the population used for generating genotyping data, and the variability in haplotype diversity across the target genomic regions. Therefore, a chosen subset of SNPs may or may not represent the haplotype architecture observed when variation from the entire sequence is considered. In that situation, more SNPs may be ultimately required to be used as a combination of tag SNPs necessary for genotyping for haplotype determination. Therefore, tag SNPs, the haplotype block, or the haplotype, may be used to identify individuals from biological samples for traits of interest. The haplotype block may, in turn, be used to identify individual polymorphic sites, or candidate disease-associated genes for developing therapeutics and diagnostics. In the present invention, a DLA class II region comprising SEQ ID NO. 1 is identified as having significant association to the risk of NME. The haplotype comprising SEQ ID NO. 1 may be further described using 35 tag SNPs, or 19 haplotype blocks, some of which can be detected by one or more tag SNPs as disclosed. As disclosed in the present invention, the haplotype, the tag SNPs and the haplotype blocks are markers or marker combinations that can be used for NME identification and treatment. The present invention also discloses a STYX region containing a haplotype associated to the risk of NME.

Other additional markers that may also be used are those genetically linked to the markers disclosed herein. These additional markers, such as, SNPs or other polymorphic markers, are in close enough proximity to have a statistically significant association with the marker disclosed herein. Such statistically significant association may be defined by linkage disequilibrium, and the subject may be placed into a group either at higher or lower risk for NME depending on the presence of the additional marker, or a close isoform thereof, that is closely linked to higher or lower risk indicating markers.

Examples of methods for detecting the haplotype blocks are described herein and other suitable methods are well known to those of skill in the art. Suitable methods for detecting haplotypes in a sample include sequence analysis, hybridization analysis using a nucleic acid probe such DNA or RNA (e.g., Northern analysis, Southern analysis, dot blot analysis), and restriction digestion, genotyping the tag SNP(s) of the haplotype in each haplotype block.

In the methods of the invention, a sample can be obtained from the individual and used in the methods to detect the presence of the haplotype blocks. The haplotype block can be detected in any sample obtained from the individual that comprises the individual's DNA. For example, a haplotype block can be detected in a tissue sample (e.g., skin, muscle, organ, placenta), a cell sample (e.g., fetal cells), a fluid sample (e.g., blood, amniotic fluid, cerebrospinal fluid, urine, lymph) and any combination thereof. Methods of obtaining such samples or extracting nucleic acid from such samples are known to those of skill in the art.

The detection of the haplotype block in the individual can be compared to a control. Suitable controls for use in the methods provided herein are apparent to those of skill in the art. For example, a suitable control can be established by assaying one or more subjects which do not have NME. Alternatively, a control can be obtained using a statistical model to obtain a control value (standard value; known standard). See, for example, models described in Knapp, R. G. and Miller M. C. (1992) Clinical Epidemiology and Biostatistics, William and Wilkins, Harual Publishing Co. Malvern, Pa., which is incorporated herein by reference.

Various parameters may be used to assess how accurately the presence or absence of a marker signifies a particular physiological or cellular characteristic. Such parameters include a positive likelihood ratio, negative likelihood ratio, odds ratio, and/or hazard ratio. In the case of a likelihood ratio, the likelihood that the presence or absence of the marker would be found in a sample with a particular cellular or physiological characteristic is compared with the likelihood that the presence or absence of the marker would be found in a sample lacking the particular cellular or physiological characteristic.

An odds ratio measures effect size and describes the amount of association or non-independence between two groups. An odds ratio is the ratio of the odds of a marker being present or absent in one set of samples versus the odds of the marker being present or absent in the other set of samples. An odds ratio of 1 indicates that the event or condition is equally likely to occur in both groups. An odds ratio greater or less than 1 indicates that presence or absence of the marker is more likely to occur in one group or the other depending on how the odds ratio calculation was set up.

A hazard ratio may be calculated by estimate of relative risk. Relative risk is the chance that a particular event will take place. It is a ratio of the probability that an event such as development or progression of a disease will occur in samples in which a particular marker is present over the probability that the event will occur in samples in which the particular marker is absent. Alternatively, a hazard ratio may be calculated by the limit of the number of events per unit time divided by the number at risk as the time interval decreases. In the case of a hazard ratio, a value of 1 indicates that the relative risk is equal in both the first and second groups; a value greater or less than 1 indicates that the risk is greater in one group or another, depending on the inputs into the calculation.

When a SNP haplotype block is identified by a SEQ ID NO, the presence of SNPs detected in a given haplotype block disclosed herein in a dog is associated with a greater risk that the dog will develop NME, if it has not yet shown symptoms of NME; whereas the absence of SNPs detected in a given haplotype block disclosed herein in a dog is associated with a lesser risk that the dog will develop NME. A nucleic acid may be termed to be specific to a haplotype block or specific to a SNP within a haplotype block. In one embodiment, a nucleic acid specific to a haplotype block or a SNP within a haplotype block contains sequence that is complementary to one of the double stranded nucleic acid sequence of at least one SNP that is within the haplotype block. Yet, in another embodiment, a nucleic acid specific to a haplotype block may be complementary to a SNP outside of the haplotype block but is associated with haplotype block. Still, in another embodiment, a nucleic acid specific to a haplotype block may be complementary to any SNPs associated with the haplotype whether the SNP is part of the haplotype block or not.

In addition to markers such as, a haplotype block, or SNPs within the haplotype block, or nucleic acids specific to the haplotype block or SNPs thereof, which are identified to be associated with a particular phenotype, a marker may be any molecular structure produced by a cell, expressed inside the cell, accessible on the cell surface, or secreted by the cell. A marker may be any protein, carbohydrate, fat, nucleic acid, catalytic site, or any combination of these such as an enzyme, glycoprotein, cell membrane, virus, cell, organ, organelle, or any uni- or multi-molecular structure or any other such structure now known or yet to be disclosed whether alone or in combination. A marker may also be called a target and the terms are used interchangeably.

A marker may be represented by the sequence of a nucleic acid from which it can be derived or any other chemical structure. Examples of such nucleic acids include miRNA, tRNA, siRNA, mRNA, cDNA, or genomic DNA sequences including complimentary sequences. Alternatively, a marker may be represented by a protein sequence. The concept of a marker is not limited to the products of the exact nucleic acid sequence or protein sequence by which it may be represented. Rather, a marker encompasses all molecules that may be detected by a method of assessing the presence of the marker, whether through direct or indirect approaches.

Examples of molecules encompassed by a marker represented by a particular sequence or structure or allele include point mutations, silent mutations, deletions, frame-shift mutations, translocations, alternative splicing derivatives, differentially methylated sequences, differentially modified protein sequences, truncations, soluble forms of cell membrane associated markers, and any other variation that results in a product that may be identified as the marker. The following non-limiting examples are included for the purposes of clarifying this concept: If expression of a specific marker in a sample is assessed by RT-PCR (Reverse Transcription Polymerase Chain Reaction), and if the sample expresses an mRNA sequence different from the sequence used to identify the specific marker by one or more nucleotides, but the marker may still be detected using RT-PCR, then the specific marker encompasses the mRNA sequence present in the sample. Alternatively, if expression of a specific marker in a sample is assessed by an antibody and the amino acid sequence of the marker in the sample differs from a sequence used to identify marker by one or more amino acids, but the antibody is still able to bind to the version of the marker in the sample, then the specific marker encompasses the amino acid sequence present in the sample.

In the present invention, the marker represented by a group of linked SNPs, “haplotype block,” “haplotype”, or gene products, including mRNA and protein, produced from the genes within the haplotype may be detected by a variety of methodologies or procedures that are well known in the art including, but not limited to, nucleic acid hybridization, antibody binding, activity assay, PCR, SI nuclease assay and via gene chip or microarray, as well as any other assay known in the art that may be used for such detection.

Detecting the presence or absence of a marker disclosed herein or a close isoform thereof may be carried out either directly or indirectly by any suitable methodology. A variety of techniques are known to those skilled in the art. Those techniques, generally, involve receiving a biological sample containing DNA or protein from the subject, and then detecting whether or not the marker or a close isoform thereof is present in the sample, and then determining the presence or absence of the marker in the sample.

The marker may be detected by direct methods by determining the presence of an allele including, but not limited to, any form of DNA sequencing such as, Sanger sequencing, next generation sequencing, pyrosequencing, sequencing by ligation, sequencing by synthesis, single molecule sequencing, pooled and barcoded DNA sequencing or any other sequencing method now known or yet to be disclosed; PCR-based methods such as, real-time PCR, quantitative PCR, reverse transcription PCR or any combination of these; allele specific ligation; comparative genomic hybridization; or any other method that allows the detection of a particular nucleic acid sequence within a sample or enables the differentiation of one nucleic acid from another nucleic acid that differs from the first nucleic acid by one or more nucleotides.

Hybridization of a SNP-specific oligonucleotide to a target polynucleotide may be performed with both entities in solution, or such hybridization may be performed when either the oligonucleotide or the target polynucleotide is covalently or non-covalently affixed to a solid support. Low stringency conditions when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent (50×Denhardt's contains per 500 ml: 5 g Ficoll (Type 400, Pharmacia), 5 g BSA (Fraction V; Sigma)) and 100 μg/ml denatured salmon sperm DNA followed by washing in a solution comprising 5×SSPE, 0.1% SDS at 42° C. when a probe of about 500 nucleotides in length is employed. Whereas high stringency conditions when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

Solid support attachment may be mediated, for example, by antibody-antigen interactions, poly-L-Lys, streptavidin or avidin-biotin interactions, salt bridges, hydrophobic interactions, chemical linkages, UV cross-linking baking, etc. SNP-specific oligonucleotides may be synthesized directly on the solid support or attached to the solid support subsequent to synthesis. Solid-supports suitable for use in detection methods of the disclosure include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the SNP-specific oligonucleotide or target nucleic acid. Detecting the nucleotide or nucleotide pair of interest may also be determined using a mismatch detection technique, including but not limited to the RNase protection method using riboprobes (Winter et al. (1985) Proc. Natl. Acad. Sci. USA 82:7575; Meyers et al. (1985) Science 230:1242) and proteins which recognize nucleotide mismatches, such as the E. coli mutS protein (Modrich (1991) Ann. Rev. Genet. 25:229-53). Alternatively, variant SNPs or variant alleles can be identified by single strand conformation polymorphism (SSCP) analysis (Orita et at. (1989) Genomics 5:874-9); Humphries et al. (1996) in MOLECULAR DIAGNOSIS OF GENETIC DISEASES, Elles, ed., pp. 321-340) or denaturing gradient gel electrophoresis (DGGE) (Wartell et al. (1990) Nucl. Acids Res. 18:2699706); Sheffield et al. (1989) Proc. Natl. Acad. Sci. USA 86:232-6).

A polymerase-mediated primer extension method may also be used to identify po lymorphism(s). Several such methods have been described in the patent and scientific literature and include the “Genetic Bit Analysis” method (WO 92/15712) and the ligase/polymerase mediated genetic bit analysis (U.S. Pat. No. 5,679,524. Related methods are disclosed in WO 91102087, WO 90/09455, WO 95/17676, and U.S. Pat. Nos. 5,302,509 and 5,945,283). Extended primers containing the complement of the polymorphism may be detected by mass spectrometry as described in U.S. Pat. No. 5,605,798. Another primer extension method is allele-specific PCR (Ruano et al. (1989) Nucl. Acids Res. 17:8392; Ruano et al. (1991) Nucl. Acids Res. 19:6877-82); WO 93/22456; Turki et al. (1995) 1. Clin. Invest. 95:1635-41). The haplotype for a gene of an individual may also be determined by hybridization of a nucleic acid sample containing one or both copies of the gene, mRNA, cDNA or fragment(s) thereof, to nucleic acid arrays and sub-arrays such as described in WO 95/112995. The arrays would contain a battery of SNP-specific or allele-specific oligonucleotides representing each of the polymorphic sites to be included in the haplotype.

Examples of indirect methods of detection include any nucleic acid detection method including the following non-limiting examples, microarray analysis, RNA in situ hybridization, RNAse protection assay, Northern blot, reverse transcriptase PCR, quantitative PCR, quantitative reverse transcriptase PCR, quantitative real-time reverse transcriptase PCR, reverse transcriptase treatment followed by direct sequencing, direct sequencing of genomic DNA, or any other method of detecting a specific nucleic acid now known or yet to be disclosed. Other examples include any process of assessing protein expression including flow cytometry, immunohistochemistry, ELISA, Western blot, and immunoaffinity chromatography, HPLC, mass spectrometry, protein microarray analysis, PAGE analysis, isoelectric focusing, 2-D gel electrophoresis, or any enzymatic assay.

In Sanger Sequencing, a single-stranded DNA template, a primer, a DNA polymerase, nucleotides and a label such as a radioactive label conjugated with the nucleotide base or a fluorescent label conjugated to the primer, and one chain terminator base comprising a dideoxynucleotide (ddATP, ddGTP, ddCTP, or ddTTP), are added to each of four reactions (one reaction for each of the chain terminator bases). The sequence may be determined by electrophoresis of the resulting strands. In dye terminator sequencing, each of the chain termination bases is labeled with a fluorescent label of a different wavelength which allows the sequencing to be performed in a single reaction.

In pyrosequencing, the addition of a base to a single stranded template to be sequenced by a polymerase results in the release of a pyrophosphate upon nucleotide incorporation. An ATP sulfurylase enzyme converts pyrophosphate into ATP, which in turn catalyzes the conversion of luciferin to oxyluciferin, which results in the generation of visible light that is then detected by a camera.

In sequencing by ligation, the molecule to be sequenced is fragmented and used to prepare a population of clonal magnetic beads, in which each bead is conjugated to a plurality of copies of a single fragment with an adaptor sequence, and alternatively, a barcode sequence. The beads are bound to a glass surface. Sequencing is then performed through 2-base encoding.

In sequencing by synthesis, randomly fragmented targeted DNA is attached to a surface. The fragments are extended and bridge amplified to create a flow cell with clusters, each with a plurality of copies of a single fragment sequence. The templates are sequenced by synthesizing the fragments in parallel. Bases are indicated by the release of a fluorescent dye correlating to the addition of the particular base to the fragment.

Other methods used to assess expression include the use of natural or artificial ligands capable of specifically binding a marker. Such ligands include antibodies, antibody complexes, conjugates, natural ligands, small molecules, nano-particles, or any other molecular entity capable of specific binding to a marker. The term “antibody” is used herein in the broadest sense and refers generally to a molecule that contains at least one antigen binding site that immunospecifically binds to a particular antigen target of interest. Antibody, thus, includes but is not limited to native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. The term thus includes full length antibodies and/or their variants as well as immunologically active fragments thereof, thus encompassing, antibody fragments capable of binding to a biological molecule (such as an antigen or receptor) or portions thereof, including but not limited to Fab, Fab′, F(ab′)2, facb, pFc′, Fd, Fv or scFv (See, e.g., CURRENT PROTOCOLS IN IMMUNOLOGY, (Colligan et al., eds., John Wiley & Sons, Inc., NY, 1994-2001). Ligands may be associated with a label such as a radioactive isotope or chelate thereof, fluorescent or nonfluorescent dye, stain, enzyme, metal, or any other substance capable of aiding a machine or a human eye from differentiating a cell expressing a marker from a cell not expressing a marker. Additionally, expression may be assessed by monomeric or multimeric ligands associated with substances capable of killing the cell. Such substances include protein or small molecule toxins, cytokines, pro-apoptotic substances, pore forming substances, radioactive isotopes, or any other substance capable of killing a cell.

The disclosure also provides sets of molecular probes for detection, including at least two probes capable of detecting, directly or indirectly, a marker disclosed herein associated with increased or decreased risk of NME, wherein the molecular probes are not associated with a microarray of greater than 1000 elements, a microarray with greater than 500 elements, a microarray with greater than 100 elements, a microarray with greater than 50 elements, or are not associated with a microarray. Such sets of two or more probes may include at least one probe capable of detecting, directly or indirectly, a marker disclosed herein associated with higher risk of developing NME and at least one other probe capable of detecting, directly or indirectly, a marker disclosed herein associated with lower risk of developing NME.

If a marker can be detected through expression level alteration, the expression of the marker in a sample may be compared to a level of expression predetermined to predict the presence or absence of a particular physiological characteristic. The level of expression may be derived from a single control or a set of controls. In one embodiment, a control does not have a particular physiological characteristic or phenotype at issue. In one embodiment, a control is a sample obtained from a healthy dog without NME. In another embodiment, a control is a tissue sample without physiological characteristic(s) of NME from a dog with NME. A control may be any sample having a previously determined expression level of the marker. Alternatively, the expression of a marker in a sample may be compared to a control that has a level of expression predetermined to signal or not signal a cellular or physiological characteristic. This level of expression may be derived from a single source of material including the sample itself or from a set of sources. Comparison of the expression of the marker in the sample to a particular level of expression results in a prediction that the sample exhibits or does not exhibit the cellular or physiological characteristic.

Prediction of a cellular or physiological characteristic includes the prediction of any cellular or physiological state that may be determined by assessing the expression or the presence or absence of a marker. Such determination includes categorizing a cell as a particular normal or diseased cell type, as having a likelihood of having or not having one or more diseases, a likelihood that a present disease will progress, remain unchanged, or regress, a likelihood that a disease will respond or not respond to a particular therapy, or any other disease outcome such as, the likelihood that a cell will move, senesce, apoptose, differentiate, metastasize, or change from any state to any other state or maintain its current state.

One type of cellular or physiological characteristic is the risk that a particular disease outcome will occur. Assessing this risk includes the performing of any type of test, assay, examination, result, readout, or interpretation that correlates with an increased or decreased probability that an individual has had, currently has, or will develop a particular disease, disorder, symptom, syndrome, or any condition related to health or bodily state. Examples of disease outcomes include, but need not be limited to survival, death, progression of existing disease, remission of existing disease, initiation of onset of a disease in an otherwise disease-free subject, or the continued lack of disease in a subject in which there has been a remission of disease. Assessing the risk of a particular disease encompasses diagnosis in which the type of disease afflicting a subject is determined. Assessing the risk of a disease outcome also encompasses the concept of prognosis. A prognosis may be any assessment of the risk of disease outcome in an individual in which a particular disease has been diagnosed. Assessing the risk further encompasses prediction of therapeutic response in which a treatment regimen is chosen based on the assessment. Assessing the risk also encompasses a prediction of overall survival after diagnosis.

The skilled artisan understands how to utilize a selected marker or a plurality of selected markers that signifies a particular physiological or cellular characteristic. In diagnosing the presence of a disease, a threshold value may be obtained by performing the assay method on samples obtained from a population of patients having a certain type of disease (e.g., NME) and from a second population of subjects that do not have the disease. In assessing disease outcome or the effect of treatment, a population of patients, all of which may develop a disease such as NME, may be followed for a period of time. After the period of time expires, the population may be divided into two or more groups. For example, the population may be divided into a first group of patients who did develop NME and a second group of patients who did not develop NME. Examples of endpoints include occurrence of one or more symptoms of disease, death or other states to which the given disease may progress. If presence of the marker in a sample population statistically significantly aligns with one group relative to the other group, the subject from which the sample was derived may be assigned a risk of having the same outcome as the patient group that differentially displays the marker.

A nucleic acid-containing sample used for various marker detection methods may be from a subject suspected of having NME. Nucleic acids may include but need not be limited to RNA, cDNA, tRNA, mitochondrial DNA, plasmid DNA, siRNA, genomic DNA, or any other naturally occurring or artificial nucleic acid molecule. The sample may be any type of sample derived from the subject, including any fluid or tissue that may contain one or more markers associated with the haplotype. Examples of sources of samples include, but are not limited to, biopsy or other in vivo or ex vivo analysis of prostate, breast, skin, muscle, fascia, brain, endometrium, lung, head and neck, pancreas, small intestine, blood, liver, testes, ovaries, colon, skin, stomach, esophagus, spleen, lymph node, bone marrow, kidney, placenta, and fetus. In some aspects of the invention, the sample comprises a fluid sample, such as peripheral blood, lymph fluid, ascites, serous fluid, pleural effusion, sputum, cerebrospinal fluid, amniotic fluid, lacrimal fluid, stool, or urine.

The subject may be any organism that may be subject to NME, MS (including mammals), and further including humans and canines.

### EXAMPLES

Various embodiments of the present teachings can be illustrated by the following non-limiting examples. The following examples are illustrative, and are not intended to limit the scope of the claims.

### Example 1

### Methods and Material

Study Population:

Purebred Pug dogs were used for the case-control genome-wide association study. Cases were verified to have NME based on clinical history and independent evaluation of hematoxylin and eosin brain sections by a veterinary neuropathologist. Cases ranged in age from 4 to 84 months (mean=18 months, median=26 months) and consisted of 11 males and 19 females. Control dogs had no evidence of neurological or autoimmune disease, ranged in age from 5 to 204 months (mean=60 months, median=48 months) at the time of sample collection and consisted of 30 males and 38 females. Control dogs were followed for 18 months after sample collection to verify that they did not develop neurological or autoimmune disease.

SNP Genotyping:

Genomic DNA was isolated using the Qiagen Gentra Puregene® Tissue Kit or Qiagen DNeasy® Blood and Tissue Kit (Qiagen N. V., Venlo, Netherlands). SNP genotyping was performed with the Illumina CanineHD Genotyping BeadChip using the Illumina BeadArray reader (Illumina Inc., San Diego, Calif.) following the manufacturer's protocol. Genomic DNA was isolated from 30 Pug dogs with histopathologically confirmed NME and 68 healthy, control Pug dogs without evidence of neurological or autoimmune disease. Genomic DNA quality was assessed with 2% agarose gel electrophoresis and quantified with fluorometric dsDNA quantification. Genome-wide association of >100,000 SNPs was performed using the Illumina Canine Infinium® HD BeadChip, and SNPs were analyzed with PLINK (found on the web at pngu.mgh.harvard.edu/purcell/plink/) with a minor allele frequency of >5% and call rate of >98%.

Statistical Analysis:

Genotyping was performed on 98 dogs, including 30 NME cases and 68 controls. Genome-wide analysis was performed with PLINK (Purcell et al. 2007). Concordance on duplicate samples was 99.96%. Only samples with a call rate of >95% were included, resulting in analysis of 28 NME cases and 66 controls. A total of 172,115 SNPs were genotyped. Classic multidimensional scaling (Purcell et al. 2007) using a call rate of >97% and MAF of >0.10 was performed on 85,366 SNPs to determine population stratification, and 21 controls that were not clustered with the main population of dogs were excluded resulting in a final population of 28 NME cases and 45 controls for analysis. These 45 control dogs ranged in age from 5 to 204 months (mean=80 months, median=48 months). Prior to analysis, 7,324 SNPs were excluded for failure to reach the call rate threshold (>95%) and 81,001 SNPs were excluded for failure to reach the MAF threshold (>0.05). In total, 86,692 SNPs were used for analysis. Bonferroni correction was applied to account for multiple hypothesis testing with a resulting P value of 5.77×10⁻⁷ across 86,692 SNPs for genome-wide significance. To further evaluate genome-wide significance, MaxT permutation testing (Purcell et al. 2007) of 100 000 permutations was applied.

### Example 2

### Identifying Disease-Associated SNPs Genome-Wide

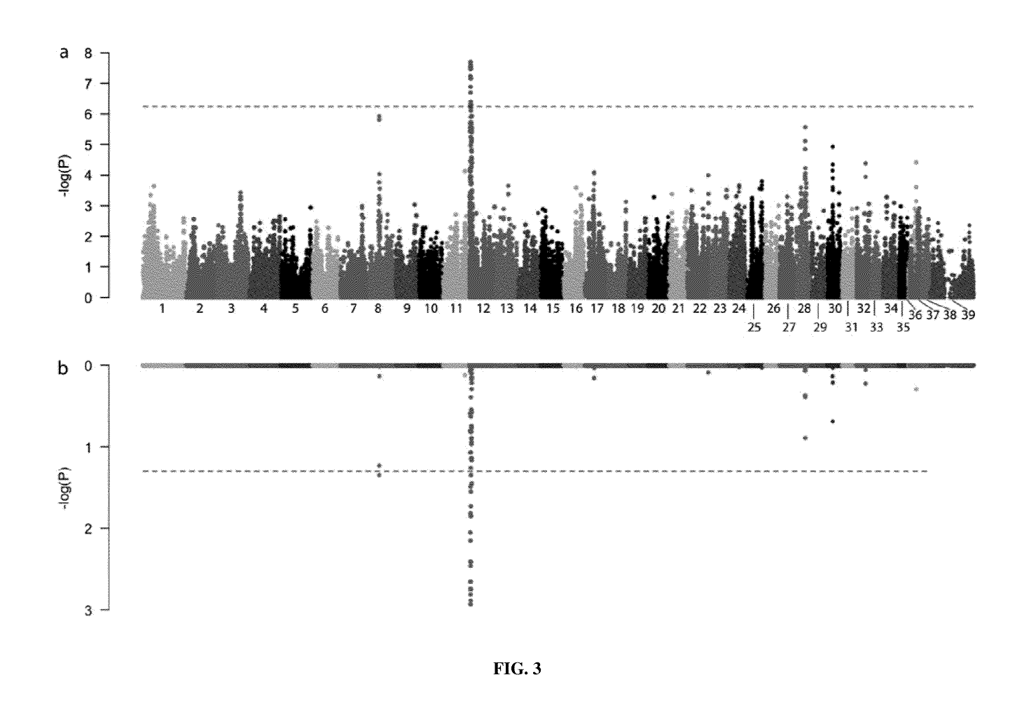

Initial genotyping was performed on 30 NME cases and 68 controls across 172,115 SNPs. After quality filtering and exclusion of population outliers, analysis of 28 NME cases and 45 controls across 86,692 SNPs identified two disease-associated loci that reached genome-wide significance with correction for multiple hypothesis testing. The strongest association was on chromosome 12 where 35 SNPs within the DLA class II region reached genome-wide significance after Bonferroni correction (raw P value for Bonferroni genome-wide significance is p<5.77×10⁻⁷) with the highest SNP having an odds ratio of 16.1 (95% CI: 4.7-55.5) (FIG. 3a and Table 1). FIG. 3 shows the genome-wide association results for 28 NME cases and 45 controls. In FIG. 3a, Fisher's exact tests were performed to compare SNP allele frequencies and negative log P values were plotted across the genome. The horizontal dotted line represents the threshold for significant association after Bonferroni correction of −log(P)>6.24 with a strong peak on chromosome 12 maintaining genome-wide significance.

TABLE 1
SNPs with genome-wide significance after Bonferroni correction

| Canine SNP | Chr | Pos | A_{R}/A_{NR} | F_{A}/F_{U} | P_{raw} | P_{genome} | OR (95% CI) | | Gene |
|---|---|---|---|---|---|---|---|---|---|
| CF2P178662 | 12 | 5166878 | A/G | 0.95/0.52 | 2.36 × 10⁻⁸ | 0.0020 | 16.1 | (4.7-55.5) | RT1-Db2 |
| BICF2S23225431 | 12 | 5217389 | G/A | 0.86/0.36 | 2.87 × 10⁻⁹ | 0.0002 | 10.5 | (4.4-25) | RT1-Db2 |
| BICF2P22942 | 12 | 5227499 | G/A | 0.86/0.36 | 2.87 × 10⁻⁹ | 0.0002 | 10.5 | (4.4-25) | RT1-Db2 |
| BICF2P194998 | 12 | 5275229 | A/T | 0.79/0.30 | 8.59 × 10⁻⁹ | 0.0007 | 8.7 | (4-19.2) | |
| rs8856588 | 12 | 5622709 | C/A | 0.79/0.30 | 8.59 × 10⁻⁹ | 0.0007 | 8.7 | (4-19.2) | COL11A2 |
| BICF2P574765 | 12 | 5710832 | A/G | 0.79/0.30 | 8.59 × 10⁻⁹ | 0.0007 | 8.7 | (4-19.2) | bing4-a |
| BICF2P1186632 | 12 | 5734305 | A/G | 0.79/0.29 | 7.24 × 10⁻⁹ | 0.0006 | 8.9 | (4.1-19.7) | TAPBP |
| BICF2P1185629 | 12 | 5791672 | G/A | 0.79/0.30 | 8.59 × 10⁻⁹ | 0.0007 | 8.7 | (4-19.2) | KIFC1 |
| BICF2P540937 | 12 | 5829667 | A/G | 0.79/0.32 | 6.27 × 10⁻⁸ | 0.0054 | 7.9 | (3.6-17.1) | CUTA |
| rs9189886 | 12 | 5843592 | G/C | 0.93/0.51 | 5.95 × 10⁻⁸ | 0.0052 | 12.4 | (4.1-37.3) | Syngap1 |
| rs9006653 | 12 | 5916360 | A/G | 0.79/0.33 | 8.07 × 10⁻⁸ | 0.0070 | 7.5 | (3.4-16.2) | Ppdpfb |
| BICF2P1200278 | 12 | 5931001 | G/A | 0.79/0.29 | 7.24 × 10⁻⁹ | 0.0006 | 8.9 | (4.1-19.7) | Ppdpfb |
| rs9125534 | 12 | 5935549 | A/G | 0.79/0.30 | 8.59 × 10⁻⁹ | 0.0007 | 8.7 | (4-19.2) | Ppdpfb |
| BICF2S23322760 | 12 | 5992526 | A/G | 0.79/0.30 | 8.59 × 10⁻⁹ | 0.0007 | 8.7 | (4-19.2) | Ppdpfb |
| rs8760645 | 12 | 6024841 | T/A | 0.79/0.30 | 8.59 × 10⁻⁹ | 0.0007 | 8.7 | (4-19.2) | Ppdpfb |
| rs9245050 | 12 | 6028685 | G/A | 0.79/0.30 | 8.59 × 10⁻⁹ | 0.0007 | 8.7 | (4-19.2) | Ppdpfb |
| BICF2P863589 | 12 | 6059850 | A/G | 0.88/0.47 | 4.83 × 10⁻⁷ | 0.0419 | 8 | (3.3-19.7) | Ppdpfb |
| BICF2P1115728 | 12 | 6064245 | C/A | 0.88/0.47 | 4.83 × 10⁻⁷ | 0.0419 | 8 | (3.3-19.7) | Ppdpfb |
| BICF2P1254053 | 12 | 6149213 | G/A | 0.84/0.35 | 7.23 × 10⁻⁹ | 0.0006 | 9.6 | (4.2-22.2) | MLN |
| BICF2P402427 | 12 | 6160615 | A/C | 0.79/0.30 | 8.59 × 10⁻⁹ | 0.0007 | 8.7 | (4-19.2) | Ggnbp1 |
| rs8694179 | 12 | 6164202 | A/G | 0.84/0.35 | 7.23 × 10⁻⁹ | 0.0006 | 9.6 | (4.2-22.2) | Ggnbp1 |
| BICF2P459960 | 12 | 6184107 | G/A | 0.84/0.35 | 7.23 × 10⁻⁹ | 0.0006 | 9.6 | (4.2-22.2) | Ggnbp1 |
| BICF2S22951431 | 12 | 6197313 | A/C | 0.79/0.30 | 8.59 × 10⁻⁹ | 0.0007 | 8.7 | (4-19.2) | Ggnbp1 |
| BICF2P1261424 | 12 | 6200280 | G/A | 0.84/0.35 | 7.23 × 10⁻⁹ | 0.0006 | 9.6 | (4.2-22.2) | Ggnbp1 |
| rs9120943 | 12 | 6218850 | A/G | 0.84/0.35 | 7.23 × 10⁻⁹ | 0.0006 | 9.6 | (4.2-22.2) | |
| rs9077055 | 12 | 6238545 | A/G | 0.84/0.35 | 7.23 × 10⁻⁹ | 0.0006 | 9.6 | (4.2-22.2) | |
| rs8677516 | 12 | 6257019 | G/A | 0.86/0.36 | 2.87 × 10⁻⁹ | 0.0002 | 10.5 | (4.4-25) | |
| BICF2P608380 | 12 | 6289014 | G/A | 0.86/0.36 | 2.87 × 10⁻⁹ | 0.0002 | 10.5 | (4.4-25) | |
| rs9132539 | 12 | 6299459 | A/G | 0.79/0.30 | 1.96 × 10⁻⁸ | 0.0017 | 8.5 | (3.9-18.6) | |
| BICF2P1340012 | 12 | 6311277 | C/A | 0.79/0.30 | 8.59 × 10⁻⁹ | 0.0007 | 8.7 | (4-19.2) | |
| BICF2P1211546 | 12 | 6320910 | A/G | 0.79/0.30 | 8.59 × 10⁻⁹ | 0.0007 | 8.7 | (4-19.2) | |
| BICF2P738783 | 12 | 6342204 | A/C | 0.79/0.29 | 6.06 × 10⁻⁹ | 0.0005 | 9.2 | (4.1-20.3) | LOC1127664 |
| BICF2P1313789 | 12 | 6653816 | A/G | 0.79/0.34 | 1.89 × 10⁻⁹ | 0.0163 | 7.1 | (3.3-15.4) | NUDT3 |
| BICF2P1380652 | 12 | 6809061 | A/G | 0.79/0.33 | 8.07 × 10⁻⁸ | 0.0070 | 7.5 | (3.4-16.2) | SPDEF |
| BICF2P1462329 | 12 | 6832252 | A/G | 0.79/0.33 | 8.07 × 10⁻⁸ | 0.0070 | 7.5 | (3.4-16.2) | SPDEF |

In Table 1, Chr stands for chromosome; Pos stands for physical position; A_{R}, stands for risk allele; A_{NR} stands for nonrisk allele; F_{A} stands for allele frequency in cases; F_{U} stands for allele frequency in controls; and OR stands for odds ratio.

Permutation testing using 100,000 permutations identified an additional four SNPs that reached genome-wide permuted significance within the DLA II locus and a second region of significance within the STYX gene on chromosome 8 (P_{raw}=2.11×10⁻⁶, P_{permuted}=0.045) with an odds ratio of 5.9 (95% CI: 2.7-12.5) (FIG. 3b and Table 2). FIG. 3b showed that MaxT 100,000 permutation testing was performed and negative log P values were plotted across the genome. The horizontal dotted line represents the threshold for significant association after permutation testing of −log(P)>1.3 with one SNP on chromosome 8 maintaining permuted significance.

TABLE 2
New SNPs discovered with genome-wide significance after permutation testing

| Canine SNP | Chr | Pos | A_{R}/A_{NR} | F_{A}/F_{U} | P_{raw} | P_{permuted} | OR [95% CI] | Gene |
|---|---|---|---|---|---|---|---|---|
| BICF2S23516667 | 8 | 31971609 | A/G | 0.73/0.32 | 2.11 × 10⁻⁶ | 0.0452 | 5.9 [2.8-12.5] | STYX |
| BICF2P639740 | 12 | 6686088 | G/A | 0.79/0.33 | 1.30 × 10⁻⁷ | 0.0283 | 7.5 [3.4-16.4] | NUDT3 |
| BICF2P535495 | 12 | 6793393 | A/G | 0.79/0.33 | 1.30 × 10⁻⁷ | 0.0453 | 7.5 [3.4-16.4] | MGC8455 |
| rs8957837 | 12 | 8822596 | C/G | 0.77/0.33 | 3.94 × 10⁻⁷ | 0.0356 | 6.7 [3.1-14.6] | LOC112577 |

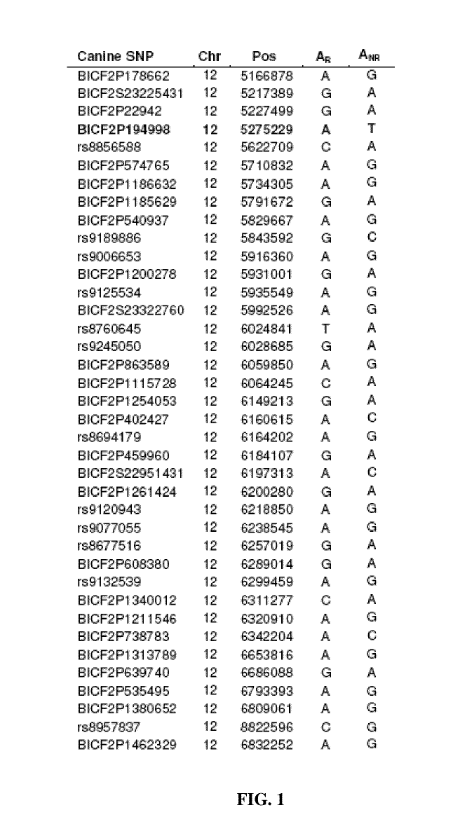

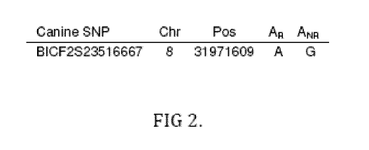

FIG. 1 showed all 38 SNPs in the DLA class II region of Chromosome 12 with disease and non-disease associated alleles listed. FIG. 2 showed the sole SNP discovered in the STYX region on Chromosome 8, with disease and non-disease associated alleles listed. To account for the fact that several of the control dogs were younger than the mean age of disease onset at the time of sample acquisition, the data was re-analyzed excluding all control dogs less than 24 months of age. Both the DLA and chromosome 8 regions remained significant with Bonferroni correction and permutation testing, respectively, but the significance was not improved by this exclusion.

### Example 3

### Identifying Disease-Associated Haplotype Blocks Over the 4.1 MB Region of DLA II on Chromosome 12

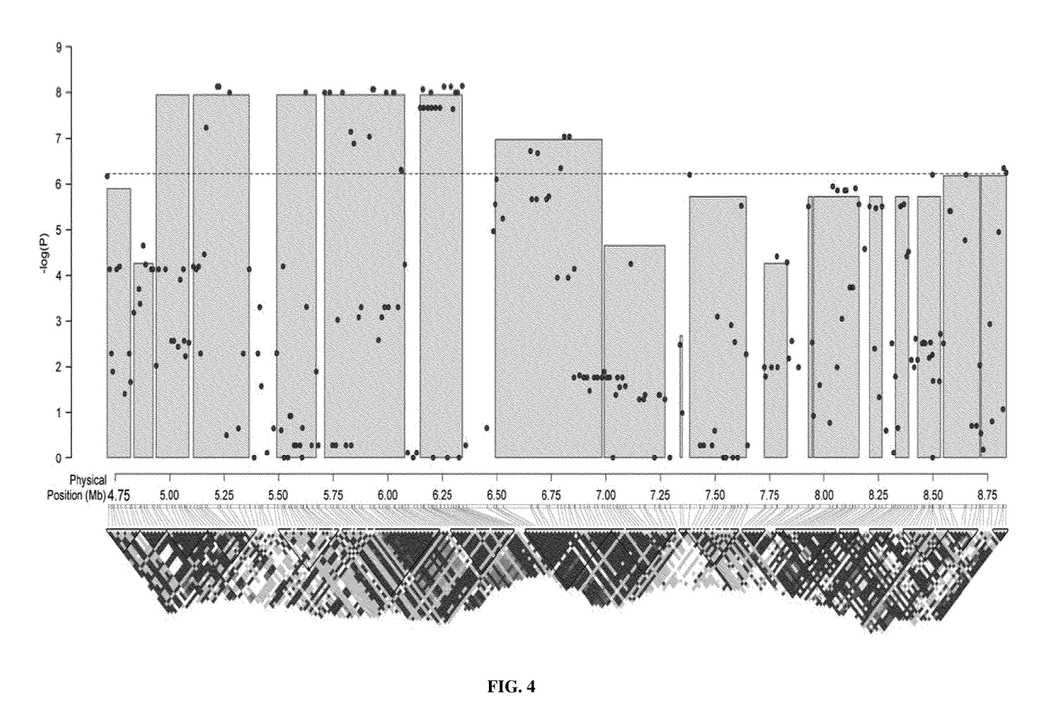

Haplotype analysis using Haploview (Barrett et al. 2005) identified 19 haplotype blocks across a 4.1 Mb region of DLA II on chromosome 12, all of which were associated with an increased risk for developing NME with P values ranging from 2.1×10⁻³ to 1.13×10⁻⁸ (FIG. 4 and Table 3). This 4.1 Mb region located from positions 4,713,392 to 8,834,652 of the genomic DNA sequence on Chromosome 12 (SEQ ID NO:1, available in the canFam2 Canine genomic sequence database) was shown in FIG. 4 with 19 haplotype blocks generated in Haploview (Gabriel et al. 2002). Negative log P values from single SNP associations were derived from genome-wide analysis after removal of population outliers and individual SNPs were plotted as circles. The horizontal dotted line represents the threshold for significant association after Bonferroni correction of −log(P)>6.24. Each haplotype is described by a group of individual tagging SNP genotypes (or alleles) that are associated with risk. The presence of some or all of these alleles will determine the risk status. For example, the tagging SNPs in Table 1 that fall in a haplotype block in Table 3 can determine the presence of that block. Based on the tagging SNPs genotype, the risk status of the haplotype block can be determined. When look at the entire 4.1 Mb region, the presence of one or more risk associated haplotype blocks can determine the risk of NME. Further, among these 23 haplotype blocks (Table 3 and Table 4), Block 4 is further described, i.e. the risk status of the haplotype block can be determined, by a combination of tagging SNPs for which the risk alleles are at position 5166878=A, 5217389=G, 5227499=G and 5275229=A. Similarly, Block 5 contains tagging SNPs for which position the risk alleles are at 5622709=C; Block 6 contains tagging SNPs for which the risk alleles are at position 5710832=A, 5734305=A, 5791672=G, 5829667=A, 5843592=G, 5916360=A, 5931001=G, 5935549=A, 5992526=A, 6024841=T, 6028685=G, 6059850=A, 6064245=C; Block 7 contains tagging SNPs for which the risk alleles are at position 6149213=G, 6160615=A, 6164202=A, 6184107=G, 6197313=A, 6200280=G, 6218850=A, 6238545=A, 6257019=G, 6289014=G, 6299459=A, 6311277=C, 6320910=A, and 6342204=A; Block 8 contains tagging SNPs for which the risk alleles are at position 6653816=A, 6686088=G, 6793393=A, 6809061=A and 6832252=A; and Block 19 contains tagging SNPs for which the risk allele is at position 8822596=C (see Table 1 and Table 3).

TABLE 3
19 Haplotype blocks generated by Haploview for DLA II
region of Chr. 12

| | | | | | Start- | SEQ |
|---|---|---|---|---|---|---|
| | | Population | Case | Control | end | ID |
| Block | Haplotype | frequency | frequency | frequency | position | NO: |
| 1 | AAGGAGAAA | 0.59 | 0.84 | 0.43 | 4713392- | 3 |
| | | | | | 4821633 | |
| 2 | AGGCAAG | 0.60 | 0.80 | 0.47 | 4836721- | 4 |
| | | | | | 4923170 | |
| 3 | GCAGGAGGGGA | 0.49 | 0.79 | 0.30 | 4938082- | 5 |
| | | | | | 5088561 | |
| 4 | AAAACAGGAAG | 0.49 | 0.79 | 0.30 | 5108726- | 6 |
| | AG | | | | 5364188 | |
| 5 | AGAAAGCGA | 0.49 | 0.79 | 0.30 | 5491709- | 7 |
| | | | | | 5672682 | |
| 6 | AAGGAGAAAGA | 0.49 | 0.79 | 0.30 | 5710832- | 8 |
| | GAGAATGGACA | | | | 6078099 | |
| 7 | GAAGAGAAGGA | 0.49 | 0.79 | 0.30 | 6149213- | 9 |
| | CAGA | | | | 6342204 | |
| 8 | GGAACAGGAAA | 0.51 | 0.79 | 0.33 | 6492201- | 10 |
| | AGAGAGGGCGA | | | | 6982375 | |
| | A | | | | | |
| 9 | CGGGAAAAGGG | 0.67 | 0.88 | 0.53 | 6992493- | 11 |
| | GAGGC | | | | 7270218 | |
| 10 | AG | 0.73 | 0.88 | 0.64 | 7338759- | 12 |
| | | | | | 7350261 | |
| 11 | AAAAAG | 0.50 | 0.75 | 0.34 | 7384390- | 13 |
| | | | | | 7643147 | |
| 12 | AGGAGA | 0.70 | 0.89 | 0.58 | 7725530- | 14 |
| | | | | | 7830733 | |
| 13 | AG | 0.50 | 0.75 | 0.34 | 7927872- | 15 |
| | | | | | 7944953 | |
| 14 | AATAAAAGAGA | 0.50 | 0.75 | 0.34 | 7950821- | 16 |
| | CGA | | | | 8158994 | |
| 15 | GGAGG | 0.50 | 0.75 | 0.34 | 8208369- | 17 |
| | | | | | 8265940 | |
| 16 | GCAAAG | 0.50 | 0.75 | 0.34 | 8327142- | 18 |
| | | | | | 8386063 | |
| 17 | GGCGAGCAAAA | 0.50 | 0.75 | 0.34 | 8429601- | 19 |
| | | | | | 8533350 | |
| 18 | ATGAACCG | 0.51 | 0.77 | 0.34 | 8546686- | 20 |
| | | | | | 8713747 | |
| 19 | GGCA | 0.51 | 0.77 | 0.34 | 8719506- | 21 |
| | | | | | 8834652 | |

Manually forcing all of these haplotype blocks into a single haplotype resulted in the creation of a 4.1 Mb haplotype containing 241 SNPs (SEQ ID NO: 1). This haplotype was common and strongly associated with an increased risk of developing NME at a case frequency of 85.1%, control frequency at 38.4% with significance p value at 7.97×10⁻⁷. The strong association of the NME disease with DLA II supports an autoimmune etiology. Haplotype analysis of the DLA II region identified a large, common block strongly associated with altered disease risk. Polygenic loci with MHC II polymorphisms show the strongest disease association, as observed between DLA II and NME association.

### Example 4

### Identifying Disease-Associated Haplotype Blocks Over the 488 kb Region of STYX on Chromosome 8

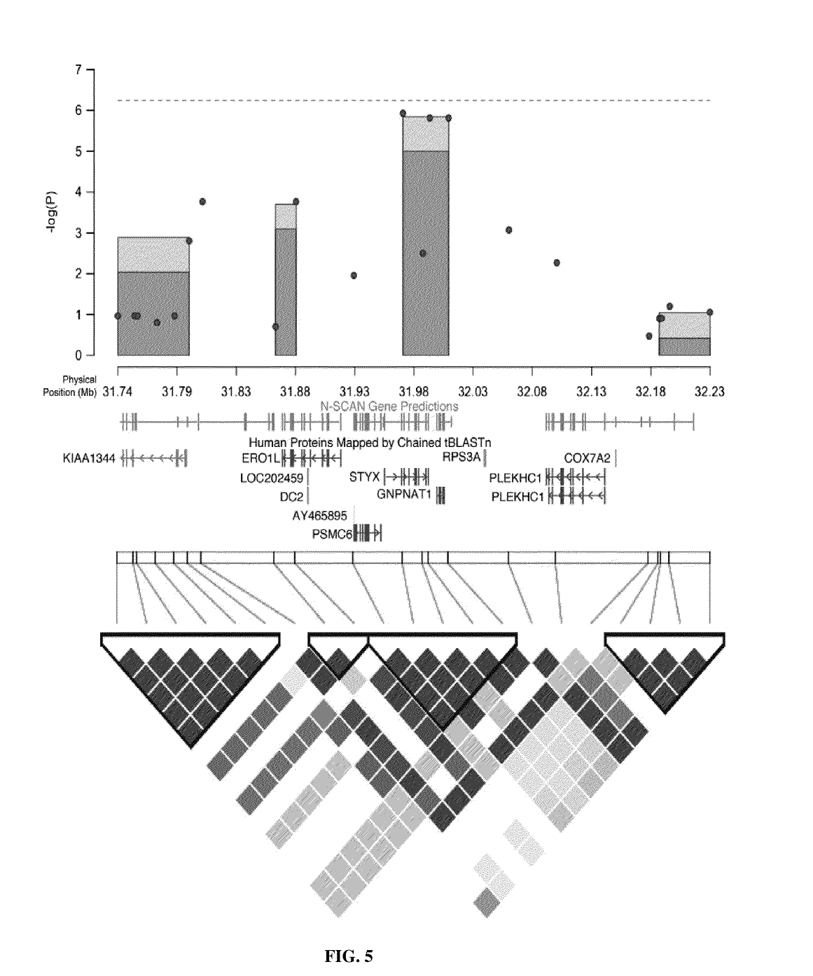

Haplotype analysis of the STYX region of chromosome 8 identified four haplotypes (FIG. 5 and Table 4). A 488 kb region located from positions 31,736,206 to 32,225,068 of the genomic DNA sequence (SEQ ID NO: 2, available in the canFam2 Canine genomic sequence database) is shown in FIG. 5 with 4 haplotype blocks generated in Haploview. Negative log P values from single SNP associations were derived from genome-wide analysis after removal of population outliers and individual SNPs were plotted as circles. The horizontal dotted line represents the threshold for significant association after Bonferroni correction of −log(P)>6.24. The additional shaded region within each haplotype block represents MaxT 100,000 permuted haplotypes.

TABLE 4
Haplotype blocks generated by Haploview for STYX region
of Chr.8

| | | | | | | | SEQ |
|---|---|---|---|---|---|---|---|
| | | Population | Case | Control | Start | End | ID |
| Block | Haplotype | frequency | frequency | frequency | position | Position | NO: |
| 20 | GAAGG | 0.74 | 0.59 | 0.83 | 31736206 | 31795128 | 22 |
| | G | | | | | | |
| 21 | AG | 0.47 | 0.27 | 0.59 | 31866373 | 31883390 | 23 |
| 22 | GGAG | 0.52 | 0.27 | 0.68 | 31971609 | 32009283 | 24 |
| 23 | AGAA | 0.50 | 0.59 | 0.44 | 32183184 | 32225068 | 25 |

The most significantly associated and common haplotype spanned the STYX and GNPNAT1 genes and was protective based on phenotype (P=1.43×10⁻⁶). This block also contained two additional haplotypes significantly associated with NME risk (p˜0.005, data not shown).

STYX, (serine/threonine/tyro sine interacting protein) is a pseudophosphatase that lacks intrinsic catalytic activity and is structurally similar to members of the dual-specificity phosphatase subfamily of protein tyrosine phosphatases. Protein tyrosine phosphatases play a key role in immune system function including lymphocyte activation, with mutations in PTPN22 having been documented in association with autoimmunity (Nang et al. 2005). STYX also is known to bind to the calcineurin substrate CRHSP-24, and calcineurin plays an important role in T cell activation. Dephosphorylation of CRHSP-24 can be prevented by administration of the immunomodulatory drugs cyclosporine and FK506.

GNPNAT1, glucosamine-phosphate N-acetyltransferase 1, is involved in amino sugar metabolism including the formation of uridine diphospho-N-acetylglucosamine (UDP-GlcNAc). UDP-GlcNAc is an important cellular metabolite necessary for the synthesis of N-linked and O-linked glycans that play important roles in normal thymic T-cell apoptosis. The disruption of GNPNAT1 is expected to lead to aberrant immune responses in NME.

The purpose of this investigation was to identify disease susceptibility loci for NME through genome-wide association (GWA) of single nucleotide polymorphisms (SNPs) in affected and non-affected Pug dogs. 170,000 SNPs, genome-wide association was performed on a small number of case and control dogs to determine disease susceptibility loci in canine necrotizing meningoencephalitis (NME), a disorder with known non-Mendelian inheritance that shares clinical similarities with atypical variants of multiple sclerosis in humans. Genotyping of 30 NME-affected Pug dogs and 68 healthy, control Pugs identified two loci associated with NME, including a region within dog leukocyte antigen class II on chromosome 12 that remained significant after Bonferroni correction. Our results support the utility of this high density SNP array, confirm that dogs are a powerful model for mapping complex genetic disorders and provide important preliminary data to support in depth genetic analysis of NME in numerous affected breeds.

### Example 5

### Identifying Disease-Associated Indels Over the 4.1 MB Region of DLA II on Chromosome 12

Next-generation whole genome sequencing utilizing the Illumina chemistry (Illumina, San Diego, Calif.) was performed to identify a functional variant within the 4.1 Mb associated haplotype on chromosome 12. Three neuropathologically verified affected pugs and two closely matched unaffected pugs over the age of 6 years were used for the sequencing. The genomes of the five dogs were sequenced to an average coverage of 13.5× and, averagely, 5 million SNPs and small indel variants per animal were identified. Among these SNPs and indels, a single base deletion causing a frame shift in the major histocompatibility complex, class II, DP beta 1 (HLA-DPB1; human leuokocyte antigen-DPB1) gene was discovered. This particular HLA-DPB1 single base deletion is located at 333 KB downstream from the most strongly associated SNP previously identified, BICF2P194998. The HLA-DPB1 deletion variant is represented by SEQ ID NO: 26 (5′-TCTTCGCGCAATTGGACAGCGCGGCGGGGGTGTTCGCGGCCGTGTCGAGCTGGGCC GAGTAACTGCCAGGAACTGGAACGTCCCCGG-3′), and a “C” is deleted at Chromosome 12 position 5608903, in comparison to the HLA-DPB1 wild type (UniProKB/Swiss-Prot No: P04440) represented by SEQ ID NO: 27 (5′-TCTTCGCGCAATTGGACAGCGCGGCGGGGGTGTTCGCGGCCGTGTCCGAGCTGGGC CGAGTAACTGCCAGGAACTGGAACGTCCCCGG-3′). This HLA-DPB1 deletion variant is in haplotype Block 5.

### Example 6

### HLA-DPB1 Deletion Variant Confirmation

The Sanger sequencing was performed on 93 pugs (26 post-mortem confirmed NME cases and 67 unaffected controls) to test if the HLA-DPB1 deletion variant was more strongly associated with NME than the previously genotyped SNPs. The P-values and odds ratios were calculated by Fisher's exact allelic tests to demonstrate the strength of NME association. Fisher's exact allelic tests showed that the HLA-DPB1 deletion was more strongly associated with NME with 1.285e-15, OR 23.2 (86.5% frequency in all cases and 21.6% in controls). The deletion variant in HLA-DPB1 is therefore the leading candidate for the functional variant within the DLA locus associated with genetic risk for NME in the pug. The deletion variant was found in 22 out of 60 investigated control dogs over 6 years of age and was found in all case dogs that were histopathologically confirmed, and such finding further indicated the non-Mendelian inheritance pattern of NME in those animals.

HLA-DPB1 belongs to the HLA class II beta chain paralogues. This class II molecule is a heterodimer consisting of an alpha (DPA) and a beta chain (DPB), both anchored in the membrane. Class II molecules are expressed in antigen presenting cells (APC) such as, B lymphocytes, dendritic cells, macrophages. HLA-DPB1 binds peptides derived from antigens that access the endocytic route of APC and presents them on the cell surface for recognition by the CD4 T-cells. HLA-DPB1 variants have been linked to multiple human disorders with autoimmune associations including lupus, multiple sclerosis, and Graves' disease. This particular HLA-DPB1 single base deletion located at 333 KB downstream from NME associated SNP BICF2P194998 enables development of further diagnostic tests, treatment approaches within the pug, and a further investigation of haplotype positive animals as possible models for human autoimmune diseases.

We claim:

1. A combination of markers for identifying NME in canine, comprising one or more haplotype blocks in a DLA class II region of canine chromosome 12, wherein the DLA class II region comprises sequence having at least 80% sequence identity with SEQ ID NO. 1, wherein SEQ ID NO.1 has a start-end position from 4713000 to 8834700.

2. The combination of markers of claim 1, wherein the one or more haplotype blocks is selected from the group consisting of haplotype blocks 1-19 and any combination thereof in Table 3.

3. The combination of markers of claim 1, wherein the DLA class II region of chromosome 12 comprises one or more tagging SNPs.

4. The combination of markers of claim 3, wherein the one or more tagging SNPs is selected from the group consisting of nucleic acid variation at canine chromosome 12 position 5166878=A or G, 5217389=G or A, 5227499=G or A, 5275229=A or T, 5622709=C or A, 5710832=A or G, 5734305=A or G, 5791672=G or A, 5829667=A or G, 5843592=G or C, 5916360=A or G, 5931001=G or A, 5935549=A or G, 5992526=A or G, 6024841=T or A, 6028685=G or A, 6059850=A or G, 6064245=C or A, 6149213=G or A, 6160615=A or C, 6164202=A or G, 6184107=G or A, 6197313=A or C, 6200280=G or A, 6218850=A or G, 6238545=A or G, 6257019=G or A, 6289014=G or A, 6299459=A or G, 6311277=C or A, 6320910=A or G, 6342204=A or C, 6653816=A or G, 6686088=G or A, 6793393=A or G, 6809061=A or G, 6832252=A or G, and 8822596=C or G;
wherein canine chromosome 12 position 5166878=A, 5217389=G, 5227499=G, 5275229=A, 5622709=C, 5710832=A, 5734305=A, 5791672=G, 5829667=A, 5843592=G, 5916360=A, 5931001=G, 5935549=A, 5992526=A, 6024841=T, 6028685=G, 6059850=A, 6064245=C, 6149213=G, 6160615=A, 6164202=A, 6184107=G, 6197313=A, 6200280=G, 6218850=A, 6238545=A, 6257019=G, 6289014=G, 6299459=A, 6311277=C, 6320910=A, 6342204=A, 6653816=A, 6686088=G, 6793393=A, 6809061=A, 6832252=A, and 8822596=C are risk alleles associated with developing Necrotizing Meningoencephalitis;
wherein the two or more risk alleles are in linkage disequilibrium with one another.

5. The combination of markers of claim 1, wherein the one or more haplotype blocks is selected from the group consisting of haplotype blocks 4-8 and 19, and any combination thereof in Table 3.

6. The combination of markers of claim 5, wherein the haplotype block 4 is identifiable by one or more tagging SNP selected from the group consisting of nucleic acid variations at canine chromosome 12 position 5166878=A or G, 5217389=G or A, 5227499=G or A, and 5275229=A or T; wherein 5166878=A, 5217389=G, 5227499=G, and 5275229=A are risk alleles associated with developing Necrotizing Meningoencephalitis.

7. The combination of markers of claim 5, wherein the haplotype block 5 is identifiable by a tagging SNP having nucleic acid variations at canine chromosome 12 position 5622709=C or A; wherein 5622709=C is a risk allele associated with developing Necrotizing Meningoencephalitis

8. The combination of markers of claim 5, wherein the haplotype block 6 is identifiable by one or more tagging SNP selected from the group consisting of nucleic acid variations at canine chromosome 12 position 5710832=A or G, 5734305=A or G, 791672=G or A, 5829667=A or G, 5843592=G or C, 5916360=A or G, 5931001=G or A, 5935549=A or G, 5992526=A or G, 6024841=T or A, 6028685=G or A, 6059850=A or G, and 6064245=C or A; wherein 5710832=A, 5734305=A, 5791672=G, 5829667=A, 5843592=G, 5916360=A, 5931001=G, 5935549=A, 5992526=A, 6024841=T, 6028685=G, 6059850=A, and 6064245=C are risk alleles associated with developing Necrotizing Meningoencephalitis.

9. The combination of markers of claim 5, wherein the haplotype block 7 is identifiable by one or more tagging SNP selected from the group consisting of nucleic acid variations at canine chromosome 12 position 6149213=G or A, 6160615=A or C, 6164202=A or G, 6184107=G or A, 6197313=A or C, 6200280=G or A, 6218850=A or G, 6238545=A or G, 6257019=G or A, 6289014=G or A, 6299459=A or G, 6311277=C or A, 6320910=A or G, 6342204=A or C; wherein 6149213=G, 6160615=A, 6164202=A, 6184107=G, 6197313=A, 6200280=G, 6218850=A, 6238545=A, 6257019=G, 6289014=G, 6299459=A, 6311277=C, 6320910=A, and 6342204=A are risk alleles associated with developing Necrotizing Meningoencephalitis.

10. The combination of markers of claim 5, wherein the haplotype block 8 is identifiable by one or more tagging SNP selected from the group consisting of nucleic acid variations at canine chromosome 12 position 6653816=A or G, 6686088=G or A, 6793393=A or G, 6809061=A or G, 6832252=A or G, wherein 6653816=A or G, 6686088=G or A, 6793393=A or G, 6809061=A or G, 6832252=A or G are risk alleles associated with developing Necrotizing Meningoencephalitis.

11. The combination of markers of claim 5, wherein the haplotype block 19 is identifiable by a tagging SNP having nucleic acid variations at canine chromosome 12 position 8822596=C or G; wherein 8822596=C is a risk allele associated with developing Necrotizing Meningoencephalitis.

12. The combination of markers of claim 1 further comprising, a haplotype block in STYX region of chromosome 8, wherein the STYX region comprises sequence having at least 80% sequence identity with SEQ ID No. 2, wherein SEQ ID No. 2 has a start-end position from 31736000 to 32225100.

13. The combination of markers of claim 12, wherein the STYX region of chromosome 8 comprises a tagging SNP having nucleic acid variation at position 31971609=A or G, wherein 31971609=A is a risk allele associated with developing Necrotizing Meningoencephalitis.

14. The combination of markers of claim 1, further comprises a HLA-DPB1 single base deletion variant at position 5608903, wherein the deletion variant comprising SEQ ID NO.26 is associated with developing Necrotizing Meningoencephalitis.

15. The combination of markers of claim 5, wherein the haplotype block 5 is identifiable by the HLA-DPB 1 single base deletion variant comprising SEQ ID NO.26.

16. The combination of markers of claim 15, wherein the haplotype block 5 is identifiable by the HLA-DPB1 single base deletion variant comprising SEQ ID NO.26 and a tagging SNP having nucleic acid variations at canine chromosome 12 position 5622709=C or A, wherein 5622709=C is a risk allele associated with developing Necrotizing Meningoencephalitis.

17. The combination of markers of claim 1, wherein the one or more haplotype blocks is detected by genotyping using PCR, sequencing, hybridization, restriction digestion, or any combination thereof.

18. The combination of markers of claim 1, wherein the canine species is selected from a group consisting of Pug, Chihuahua, West Highland White Terrier, Pekingese, Labrador Retriever, Golden Retriever, Beagle, German Shepherd, Dachshund, Yorkshire Terrier, Boxer, Poodle, Shih Tzu, Miniature Schnauzer, Pomeranian, Cocker Spaniel, Rottweiler, Bulldog, Shetland Sheepdog, Boston Terrier, Miniature Pinscher, Maltese, German Shorthaired Pointer, Doberman Pinscher, Siberian Husky, Pembroke Welsh Corgi, Basset Hound, Bichon Frise, and other currently recognized and or yet to be recognized breeds.

19. A combination of markers for identifying NME in canine, comprising a HLA-DPB1 single base deletion variant at position 5608903 of canine chromosome 12, wherein the deletion variant comprising SEQ ID NO.26 is associated with developing Necrotizing Meningoencephalitis.

20. The combination of markers of claim 19, further comprises one or more tagging SNPs selected from the group consisting of nucleic acid variations at canine chromosome 12 position 5166878=A or G, 5217389=G or A, 5227499=G or A, 5275229=A or T, 5622709=C or A, 5710832=A or G, 5734305=A or G, 5791672=G or A, 5829667=A or G, 5843592=G or C, 5916360=A or G, 5931001=G or A, 5935549=A or G, 5992526=A or G, 6024841=T or A, 6028685=G or A, 6059850=A or G, 6064245=C or A, 6149213=G or A, 6160615=A or C, 6164202=A or G, 6184107=G or A, 6197313=A or C, 6200280=G or A, 6218850=A or G, 6238545=A or G, 6257019=G or A, 6289014=G or A, 6299459=A or G, 6311277=C or A, 6320910=A or G, 6342204=A or C, 6653816=A or G, 6686088=G or A, 6793393=A or G, 6809061=A or G, 6832252=A or G, and 8822596=C or G;
wherein 5166878=A, 5217389=G, 5227499=G, 5275229=A, 5622709=C, 5710832=A, 5734305=A, 5791672=G, 5829667=A, 5843592=G, 5916360=A, 5931001=G, 5935549=A, 5992526=A, 6024841=T, 6028685=G, 6059850=A, 6064245=C, 6149213=G, 6160615=A, 6164202=A, 6184107=G, 6197313=A, 6200280=G, 6218850=A, 6238545=A, 6257019=G, 6289014=G, 6299459=A, 6311277=C, 6320910=A, 6342204=A, 6653816=A, 6686088=G, 6793393=A, 6809061=A, 6832252=A; and 8822596=C are risk alleles associated with developing Necrotizing Meningoencephalitis;
wherein the two or more risk alleles are in linkage disequilibrium with one another.

21. A method of classifying a subject to an NME disease risk group, comprising:
receiving a nucleic acid-containing sample from the subject;
detecting the presence of a combination of markers comprising one or more haplotype blocks and tagging SNPs in a DLA class II region of canine chromosome 12 comprising sequences having at least 80% sequence identity with SEQ ID No. 1, wherein SEQ ID No.1 has start-end position from 4713000 to 8834700; and
classifying the subject into a risk group based upon the presence of at least one haplotype block, or classifying the subject into a non-risk group based upon the absence of any haplotype block.

22. The method of claim 21, wherein the one or more haplotype blocks is selected from the group consisting of haplotype blocks 1-19 and any combination thereof in Table 3.

23. The method of claim 21, wherein the one or more tagging SNPs is selected from the group consisting of nucleic acid variation at canine chromosome 12 position 5166878=A or G, 5217389=G or A, 5227499=G or A, 5275229=A or T, 5622709=C or A, 5710832=A or G, 5734305=A or G, 5791672=G or A, 5829667=A or G, 5843592=G or C, 5916360=A or G, 5931001=G or A, 5935549=A or G, 5992526=A or G, 6024841=T or A, 6028685=G or A, 6059850=A or G, 6064245=C or A, 6149213=G or A, 6160615=A or C, 6164202=A or G, 6184107=G or A, 6197313=A or C, 6200280=G or A, 6218850=A or G, 6238545=A or G, 6257019=G or A, 6289014=G or A, 6299459=A or G, 6311277=C or A, 6320910=A or G, 6342204=A or C, 6653816=A or G, 6686088=G or A, 6793393=A or G, 6809061=A or G, 6832252=A or G, and 8822596=C or G;
wherein 5166878=A, 5217389=G, 5227499=G, 5275229=A, 5622709=C, 5710832=A, 5734305=A, 5791672=G, 5829667=A, 5843592=G, 5916360=A, 5931001=G, 5935549=A, 5992526=A, 6024841=T, 6028685=G, 6059850=A, 6064245=C, 6149213=G, 6160615=A, 6164202=A, 6184107=G, 6197313=A, 6200280=G, 6218850=A, 6238545=A, 6257019=G, 6289014=G, 6299459=A, 6311277=C, 6320910=A, 6342204=A, 6653816=A, 6686088=G, 6793393=A, 6809061=A, 6832252=A, and 8822596=C are risk alleles associated with developing Necrotizing Meningoencephalitis;
wherein the two or more risk alleles are in linkage disequilibrium with one another.

24. The method of claim 21, wherein the canine species is selected from a group consisting of Pug, Chihuahua, West Highland White Terrier, Pekingese, Labrador Retriever, Golden Retriever, Beagle, German Shepherd, Dachshund, Yorkshire Terrier, Boxer, Poodle, Shih Tzu, Miniature Schnauzer, Pomeranian, Cocker Spaniel, Rottweiler, Bulldog, Shetland Sheepdog, Boston Terrier, Miniature Pinscher, Maltese, German Shorthaired Pointer, Doberman Pinscher, Siberian Husky, Pembroke Welsh Corgi, Basset Hound, Bichon Frise, and other currently recognized and or yet to be recognized breeds.
